(19) United States
(12) Patent Application Publication
(10) Pub. No.: US 2024/0108501 A1
(43) Pub. Date: Apr. 4, 2024

(54) APPARATUS AND METHODS FOR DELIVERY OF CELL COMPOSITIONS

(71) Applicant: FUJIFILM Cellular Dynamics, Inc., Madison, WI (US)

(72) Inventors: Tianran LIU, Madison, WI (US); Randy LEARISH, Madison, WI (US); Cayla THOMPSON, Madison, WI (US); Matthew STERNFELD, Madison, WI (US); Michael PHILLIPS, Madison, WI (US); Virginia MATTIS, Madison, WI (US); Lucas CHASE, Madison, WI (US); Erik BROWN, Sarasota, FL (US)

(73) Assignee: FUJIFILM Cellular Dynamics, Inc., Madison, WI (US)

(21) Appl. No.: 18/479,052

(22) Filed: Sep. 30, 2023

Related U.S. Application Data
(60) Provisional application No. 63/412,195, filed on Sep. 30, 2022.

(51) Int. Cl. A61F 9/00; A61K 35/30

(52) U.S. Cl. CPC A61F 9/0017; A61K 35/30; A61B 2017/00969

(57) ABSTRACT

Provided for herein is a method of cell transplantation using an injection attachment having a hub containing a plurality of cells and a cannula extending from a distal end of the hub. The method includes orienting the injection attachment such that the cannula points downwardly until the plurality of cells settle together proximate the distal end, inserting the cannula into a tissue of a subject, and injecting the volume of cells into the tissue of the subject using the injection attachment.

### PRIORITY CLAIM

This application claims benefit of priority to U.S. Provisional Application Ser. No. 63/412,195 filed Sep. 30, 2022, the entire contents of which are hereby incorporated by reference.

### FIELD

The present disclosure relates generally to the field of cell therapy. More particularly, it concerns an apparatus and method for the delivery of cell compositions to a subject and methods of use thereof.

### BACKGROUND

Cell therapy comprises the transfer of autologous or allogeneic cellular material into a patient for medical purposes. Cell-based therapy involves the delivery of cells to circulation or tissues for the treatment of a multitude of diseases. Today, cell therapy continues to evolve with ongoing investigations for clinical safety and efficacy. Cell therapy combines stem cell- and non-stem cell-based unicellular or multicellular therapies. It typically employs autologous or allogeneic cells; might involve genetic engineering or manipulations in formulation; and can be administered topically or as injectables, infusions, bioscaffolds, or scaffold-free systems. Cell therapy spans multiple therapeutic areas, such as regenerative medicine, immunotherapy, and cancer therapy. Currently, most cell therapies are in early stages of development (phase 1/2), with several exceptions being either a current best practice in specific settings (e.g., bone marrow/stem cell transplants, hepatocyte transplantation, skin equivalents), or approved for specific indications, such as PROVENGE® (sipuleucel-T), LAVIV® (azficel-T), MACI® (autologous cultured chondrocytes on porcine collagen), and KYMRIAH™ (tisagenlecleucel) among others (El-Kadiry et al., Front. Med., 8; 2021).

Pluripotent stem cells (PSCs), which include human embryonic stem cells (hESCs) and induced pluripotent stem cell (iPSC), have been used to study development of disease processes, and as potential therapies in multiple organ systems. In recent years, there has been increasing interest in the use of PSC-based transplantation to treat disorders of the retina in which retinal cells have been functionally damaged or lost through degeneration. Preclinical trials in animal models of retinal diseases have shown improvement in visual outcomes following subretinal transplantation of PSC-derived photoreceptors or retinal pigment epithelium (RPE) cells. Death of retinal photoreceptor cells (PRs; rods and cones) because of inherited retinal disease (IRD) or injury is a leading cause of untreatable blindness worldwide. IRD (also referred to as photoreceptor degenerative diseases) can present as loss of either primarily rod PRs, primarily cone PRs, or simultaneous loss of both rod and cone PRs. Once lost, PRs cannot regenerate and treatment options for these patients are limited or nonexistent. These patients may be treated with allogeneic human induced pluripotent stem cell (iPSC)-derived photoreceptor precursor cells (iPRPs) as a cell-based PR replacement therapy for IRD.

For cell-based therapies, successful translation of preclinical research into clinical practice requires a means of cell delivery effective at the scale of the human patient. There are a number of injection strategies for cell-based therapies. Direct injection encompasses the injection of cells into the body using a needle and a syringe or through a delivery device such as a port or catheter/reservoir system. Intravenous injection is the most easily accessible route of injection for patients, with infusion into the bloodstream. Transplanted cells are usually delivered with a guided straight cannula or needle. In general, this cannula is coupled to some form of syringe used to dispense a cellular suspension.

Issues with delivery of cell therapy include unintended deposition of cells to non-target locations, unpredictable cell dosing at the intended target site, and even substantial loss of cell suspension. Cell delivery cannulas are generally connected to an external syringe via a Luer lock or similar coupling mechanism. This design has several disadvantages. First, for most syringes, small movements of the plunger dispense relatively large volumes, making the delivery of small, precise doses of cell suspension difficult to achieve manually. To address this issue, incorporation of mechanical or electrical drives to control the translational movements of the plunger could prove beneficial. Second, mechanical forces at the transition point between syringe and catheter can damage cells. The inner diameter of a syringe is typically larger than that of an injection cannula. Cells and fluid thus experience a considerable increase in linear velocity as they pass from syringe to cannula. This generates differential velocities along the length of a cell, known as an extensional force, which is thought to be a significant contributor to cell injury during injection. Furthermore, cells are also exposed to shear stresses as cells and fluid in the middle of a cannula travel at a higher velocity than those at the outer boundary (Potts et al., Surg Neurol Inst., 4(S22-S30):2013).

Many cell therapy approaches aim to deliver high-density single-cell suspensions to diseased or injured sites in the body. However, cell aggregates maintain viability, cellular activity, and phenotype beyond that of single cells, even in non-adhesive matrices, enabling delivery of higher cell densities with enhanced proliferative and differentiation capacity. Thus, there is an unmet need for improved methods and devices for the delivery of cell therapy, particularly cell aggregates, without damage or loss of cells and delivery of precise high-density doses to the target tissue.

### SUMMARY

In a first embodiment, the present disclosure provides a method for cell transplantation comprising injecting cells to a tissue of a subject using a cell delivery apparatus comprising a cannula with a funnel shaped hub.

In particular aspects, the present hub has essentially no catch points that could increase shear or turbulence as the cells move through the hub. In particular aspects, the hub is mostly smooth as the cells transit through the hub and into the cannula shaft. In certain aspects, the reduction in turbulence or shear helps improve cell viability while catch points cause cell waste and damage. In specific aspects, the present hub has essentially no catch points and is mostly smooth to allow ejection of increased cellular material (i.e., less material retained in the hub) as compared to a hub without the funnel shape.

In certain aspects, the cannula hub is less than 30 mm in length. In some aspects, the cannula comprises a cannula tip that is 30 gauge or smaller. In particular aspects, the cannula tip is blunt. In other aspects, the cannula tip is sharp. In some aspects, the cannula tip is further defined as a needle tip. In some aspects, the cannula tip is a 34, 33, 32, 31, or 30 gauge cannula tip. In some aspects, the cannula tip is a 33 gauge cannula tip. In certain aspects, the cells are loaded into the cannula with a cannula tip that is 30 gauge or smaller. In some aspects, the cells are loaded into the cannula with a 33 gauge cannula tip. In some aspects, the cells are loaded into the cannula and injected to the tissue of said subject with the same cannula tip. In certain aspects, the cannula tip is not changed between loading the cells into the cannula and the cells being injected to the tissue (e.g., the eye) of said subject.

In some aspects, the cannula tip is made of a flexible polyimide material or metal. In some aspects, the cannula tip is flexible. In certain aspects, the cannula tip is rigid.

In certain aspects, the hub comprises a one-way check valve. In certain aspects, the hub comprises a coupling mechanism. In some aspects, the coupling mechanism is a luer lock. In certain aspects, the luer lock is connected to a dosing mechanism. A controlled pressure may be applied to a dosing mechanism to deliver the dose. In some aspects, the doing mechanism is a syringe. In some aspects, the syringe is a microinjection syringe. In certain aspect, the syringe is further connected to tubing. In some aspects, the tubing is connected to a pressure control system. In certain aspects, the cells are delivered from the cell delivery apparatus at a controlled pressure. In some aspects, the cells are not redistributed or resuspended prior to loading to the cell delivery apparatus. In some aspects, the cells are redistributed or resuspended prior to loading to the cell delivery apparatus. In certain aspects, the cells are redistributed or resuspended by vortexing or manual agitation.

In some aspects, the cells are injected to an eye of said subject. In certain aspects, the cells are injected subretinally. In some aspects, the cells are further defined as cell aggregates. In certain aspects, the cells are further defined as single cells. In some aspects, the cells are in a formulation buffer. In some aspects, the formulation buffer is a balanced salt solution. In particular aspects, the balanced salt solution further comprises benzonase and/or human serum albumin. In some aspects, the cell aggregates are not in suspension when injected to the tissue of said subject. In certain aspects, the cells are present in the cannula for at least 1 minute (e.g., at least 1, 2, 3, 4, 5, 6, 7, 8, 9, 10, 11, 12, 13, 14, 15, 16, 17, 18, 19, or 20 minutes, such as 1-3, 2-4, 3-5, 3-6, 4-7, 5-8, 6-9, 7-10, 8-11, 9-12, 10-13, 11-14, 12-15, 13-16, 15-17, 16-18, 17-19, 18-20, 1-5, 5-10, 10-15, or 15-20 minutes) between loading and injecting to allow aggregate settling in the cannula hub. In certain aspects, the cells are present in the cannula for at least 1 minute (e.g., at least 1, 2, 3, 4, 5, 6, 7, 8, 9, 10, 11, 12, 13, 14, 15, 16, 17, 18, 19, or 20 minutes, such as 1-3, 2-4, 3-5, 3-6, 4-7, 5-8, 6-9, 7-10, 8-11, 9-12, 10-13, 11-14, 12-15, 13-16, 15-17, 16-18, 17-19, 18-20, 1-5, 5-10, 10-15, or 15-20 minutes) between loading and injecting to allow cell, such as single cells, settling in the cannula hub. In particular aspects, the cannula is pointed down to allow cell setting in the cannula hub.

In certain aspects, the cell aggregates are photoreceptor precursor cell aggregates. In certain aspects, the cells are retinal progenitor cells (RPEs) and/or photoreceptor precursor cells. In some aspects, the cell aggregates are RPEs and/or photoreceptor precursor cells. In some aspects, the cells are injected in a volume of less than 200 uL. In some aspects, the cells are injected in a volume less than 100 uL. In some aspects, the cells are injected in a volume of about 50 uL (e.g., about 25-50 uL, 50-75 uL, 75-100 uL, 100-150 uL, or 150-200 uL, such as about 25, 50, 75, 100, 125, 150, 175, or 200 uL). In some aspects, at least 100,000, 250,000, 500,000, or 1 million cell are injected. In certain aspects, at least 2 million, 3 million, 4 million, 5 million, 6 million, 7 million, 8 million, 9 million, 10 million, or 15 million cells are injected. In some aspects, at least 25% of the cells that are loaded into the cell delivery apparatus are injected to the tissue of said subject. In some aspects, at least 30% (e.g., 35%, 40%, 45%, 50%, 60%, 70%, 80%, 90%, 95%, 99%, or more) of the cells that are loaded into the cell delivery apparatus are injected to the tissue of said subject.

A further embodiment provides a method for treating an ocular condition in a subject comprising cell transplantation in the eye of the subject using a cell delivery apparatus comprising a cannula with a funnel shaped hub. In certain aspects, the cells are injected subretinally. In some aspects, the cannula hub is less than 30 mm in length. In certain aspects, the cannula comprises a cannula tip that is 30 gauge or smaller. In some aspects, the cannula tip is blunt. In certain aspects, the canula tip is sharp. In some aspects, the cannula tip is further defined as a needle tip. In certain aspects, the cannula tip is a 34, 33, 32, 31, or 30 gauge cannula tip. In particular aspects, the cannula tip is a 33 gauge cannula tip. In some aspects, the cells are loaded into the cannula with a cannula tip that is 30 gauge or smaller. In certain aspects, the cells are loaded into the cannula with a 33 gauge cannula tip.

In some aspects, the cells are loaded into the cannula and injected to the eye of said subject with the same cannula tip. In some aspects, the cannula tip is not changed between loading into the cannula and injected to the eye of said subject. In some aspects, the cannula tip is made of a flexible polyimide material or metal. In some aspects, the cannula tip is flexible. In certain aspects, the cannula tip is rigid.

In certain aspects, the hub comprises a one-way check valve. In some aspects, the hub comprises a coupling mechanism. In some aspects, the coupling mechanism is a luer lock. In certain aspects, the luer lock is connected to a dosing mechanism. In some aspects, the doing mechanism is a syringe. In some aspects, the syringe is a microinjection syringe. In certain aspects, the syringe is further connected to tubing. In some aspects, the tubing is connected to a pressure control system.

In particular aspects, the cells are delivered from the cell delivery apparatus at a controlled pressure. In some aspects, the cells are not redistributed or resuspended prior to loading to the cell delivery apparatus. In certain aspects, the cells are redistributed or resuspended prior to loading to the cell delivery apparatus. In some aspects, the cells are redistributed or resuspended by vortexing or manual agitation. In some aspects, the cells are injected subretinally.

In particular aspects, the cells are further defined as cell aggregates. In some aspects, the cells are further defined as single cells. In some aspects, the cells are in a formulation buffer. In certain aspects, the formulation buffer is a balanced salt solution. In some aspects, the balanced salt solution further comprises benzonase and/or human serum albumin In particular aspects, the cell aggregates are not in suspension when injected to the tissue of said subject. In some aspects, the cells are present in the cannula for at least 1 minute (e.g., at least 1, 2, 3, 4, 5, 6, 7, 8, 9, 10, 11, 12, 13, 14, 15, 16, 17, 18, 19, or 20 minutes, such as 1-3, 2-4, 3-5, 3-6, 4-7, 5-8, 6-9, 7-10, 8-11, 9-12, 10-13, 11-14, 12-15, 13-16, 15-17, 16-18, 17-19, 18-20, 1-5, 5-10, 10-15, or 15-20 minutes) minutes between loading and injecting to allow cell (e.g., aggregate) settling in the cannula hub. In particular aspects, the cannula is pointed down to allow cell setting in the cannula hub. In some aspects, the cell aggregates are photoreceptor precursor cell aggregates. In certain aspects, the cells are retinal progenitor cells (RPEs) and/or photoreceptor precursor cells. In some aspects, the cell aggregates are RPEs and/or photoreceptor precursor cells. In certain aspects, the cells are injected in a volume of less than 200 uL In some aspects, the cells are injected in a volume less than 100 uL In some aspects, the cells are injected in a volume of about 50 uL (e.g., about 25-50 uL, 50-75 uL, 75-100 uL, 100-150 uL, or 150-200 uL, such as about 25, 50, 75, 100, 125, 150, 175, or 200 uL). In particular aspects, at least 100,000, 250,000, 500, 000, or 1 million cell are injected. In certain aspects, at least 2 million, 3 million, 4 million, 5 million, 6 million, 7 million, 8 million, 9 million, 10 million, or 15 million cells are injected. In some aspects, at least 25% of the cells that are loaded into the cell delivery apparatus are injected to the eye of said subject. In some aspects, the ocular condition is an injury, inherited retinal disease, age-related macular degeneration (AMD), inherited macular degeneration, Stargardt's macular dystrophy, Best disease, choroideremia, diabetic retinopathy, retinal vascular disease, damage caused by retinopathy of prematurity (ROP), or viral infection of the eye. Injuries to the eye may include bruises, punctures, scratches, penetrating injury, perforation injury, or injury due to an intraocular foreign body.

A further embodiment provides a cannula apparatus for cell translation comprising a tubular hub, wherein the tubular hub comprises: an outer wall; an inner wall; a first end; and a second end, wherein the first end has a funnel shape sloped towards the second end. In some aspects, the inner diameter of the tubular hub is decreasing as measured from the first end towards the second end. In certain aspects, the hub has essentially no catch points. In some aspects, the hub has a smooth surface.

In particular aspects, the present hub has essentially no catch points that could increase shear or turbulence as the cells move through the hub. In certain aspects, the hub is mostly smooth as the cells transit through the hub and into the cannula shaft. In particular aspects, the reduction in turbulence or shear helps improve cell viability while catch points cause cell waste and damage. In specific aspects, the present hub has essentially no catch points and is mostly smooth to allow ejection of increased cellular material (i.e., less material retained in the hub) as compared to a hub without the funnel shape.

In some aspects, the first end is attached to a cannula tip. In certain aspects, the cannula tip is blunt. In some aspects, the cannula tip is sharp. In some aspects, the cannula tip is further defined as a needle tip.

In some aspects, the tubular hub is less than 30 mm in length. the cannula tip is 30 gauge or smaller. In certain aspects, the cannula tip is a 34, 33, 32, 31, or 30 gauge cannula tip. In some aspects, the cannula tip is a 33 gauge cannula tip. In some aspects, the second end comprises a one-way check valve integrated in the hub that prevent back flow.

In particular aspects, the valve is further connected to tubing. In some aspects, the cannula tip has an outer diameter range of about 0.30 mm to about 0.18 mm. In some aspects, the cannula tip is made of a flexible polyimide material or metal. In certain aspects, the cannula tip has a length of 1 mm, 2 mm, 3 mm, 4 mmm, or 5 mm.

In some aspects, the cannula tip is flexible. In certain aspects, the cannula tip is rigid. In some aspects, the cannula is attached to an injection device. In certain aspects, the injection device is a syringe. In certain aspects, the syringe comprises a handle to adjust the flow path of fluid. In particular aspects, the syringe is a microinjection syringe. In some aspects, the syringe comprises a fluid volume of about 1 mL. In some aspects, the tubular hub comprises a fluid volume less than 200 uL. In particular aspects, the tubular hub comprises a fluid volume less than 100 uL. In some aspects, the tubular hub comprises a fluid volume of about 50 uL (e.g., about 25-50 uL, 50-75 uL, 75-100 uL, 100-150 uL, or 150-200 uL, such as about 25, 50, 75, 100, 125, 150, 175, or 200 uL). In some aspects, the cannula apparatus is further defined as a subretinal delivery apparatus.

Further embodiments provide the use of the present apparatus in the delivery of cells to a tissue of a subject. Also provided herein is the use of the present apparatus in the treatment of an ocular condition in a subject comprising administering an effective amount of cells to an eye of said subject. In some aspects, the cells are loaded into the cannula and injected to the eye of said subject with the same cannula tip. In certain aspects, the cannula tip is not changed between loading into the cannula and injected to the eye of said subject. In some aspects, the cells are delivered from the cell delivery apparatus at a controlled pressure. In certain aspects, the cells are not redistributed or resuspended prior to loading to the cell delivery apparatus. In some aspects, the cells are redistributed or resuspended prior to loading to the cell delivery apparatus. In some aspects, the cells are redistributed or resuspended by vortexing or manual agitation. In certain aspects, the cells are injected subretinally. In some aspects, a pre-bleb is created with a different cannula prior to injection of the cells with the funnel shaped hub. In some aspects, the cells are further defined as cell aggregates. In certain aspects, the cells are further defined as single cells. In some aspects, the cells are in a formulation buffer. In particular aspects, the formulation buffer is a balanced salt solution. In specific aspects, the balanced salt solution further comprises benzonase and/or human serum albumin.

In certain aspects, the cell aggregates are not in suspension when injected to the tissue of said subject. In some aspects, the cells are present in the cannula for at least 5 minutes between loading and injecting to allow aggregate settling in the cannula hub. In some aspects, the cells are present in the cannula for at least 1 minute (e.g., at least 1, 2, 3, 4, 5, 6, 7, 8, 9, 10, 11, 12, 13, 14, 15, 16, 17, 18, 19, or 20 minutes, such as 1-3, 2-4, 3-5, 3-6, 4-7, 5-8, 6-9, 7-10, 8-11, 9-12, 10-13, 11-14, 12-15, 13-16, 15-17, 16-18, 17-19, 18-20, 1-5, 5-10, 10-15, or 15-20 minutes) minutes between loading and injecting to allow cell, such as single cells, settling in the cannula hub. In particular aspects, the cannula is pointed down to allow cell setting in the cannula hub. In certain aspects, the cell aggregates are photoreceptor precursor cell aggregates. In certain aspects, the cells are retinal progenitor cells (RPEs) and/or photoreceptor precursor cells. In some aspects, the cell aggregates are RPEs and/or photoreceptor precursor cells. In some aspects, the cells are injected in a volume of less than 200 uL In some aspects, the cells are injected in a volume less than 100 uL In certain aspects, the cells are injected in a volume of about 50 uL (e.g., about 25-50 uL, 50-75 uL, 75-100 uL, 100-150 uL, or 150-200 uL, such as about 25, 50, 75, 100, 125, 150, 175, or 200 uL). In some aspects, at least 100,000, 250,000, 500,000, or 1 million cells are injected. In certain aspects, at least 2 million, 3 million, 4 million, 5 million, 6 million, 7 million, 8 million, 9 million, 10 million, or 15 million are injected. In certain aspects, at least 25% of the cells that are loaded into the cell delivery apparatus are injected to the eye of said subject. In some aspects, the ocular condition is inherited retinal disease, age-related macular degeneration (AMD), inherited macular degeneration, Stargardt's macular dystrophy, Best disease, choroideremia, diabetic retinopathy, retinal vascular disease, damage caused by retinopathy pf prematurity (ROP), or viral infection of the eye.

Further provided herein is a kit comprising the apparatus of the present embodiments and aspects thereof and cells.

Further provided herein is a method of cell transplantation using an injection attachment having a hub containing a plurality of cells and a cannula extending from a distal end of the hub, the method comprising: orienting the injection attachment such that the cannula points downwardly until the plurality of cells settle together proximate the distal end; inserting the cannula into a tissue of a subject; and injecting the plurality of cells into the tissue of the subject using the injection attachment.

In certain embodiments, the hub includes a funnel shaped inner surface extending between the distal end and a proximal end. In certain embodiments, the inner surface is tapers inward from the proximal end to the distal end such that a diameter of the inner surface is smaller at the distal end than the diameter of the inner surface at the proximal end. In certain embodiments, the inner surface of the hub does not overlap with the cannula.

In certain embodiments, at least 90% of the cells settle together proximate the distal end. In certain embodiments, the injection attachment is oriented such that the cannula points downwardly for at least 2 minutes. In certain embodiments, the injection attachment is held at an angle greater than 45 degrees while injecting the plurality of cells into the tissue of the subject.

The method may further include: providing a plurality of cells to the injection attachment such that the hub is prefilled. In certain embodiments, the plurality of cells are drawn in through the distal end of the cannula to the hub prior to an injection and injected through the cannula. In certain embodiments, the cannula is not changed between providing the plurality of cells into the hub and injecting the cells into the tissue of the subject.

In certain embodiments, the cannula comprises a cannula shaft and a cannula tip extending from a distal end thereof. In certain embodiments, the cannula tip is a 33 gauge cannula tip. In certain embodiments, the cannula tip is blunt ended. In certain embodiments, the tissue is retinal tissue. In certain embodiments, the cells are stem cells.

Further provided herein is a method of cell transplantation in an eye using an injection attachment having a hub containing a plurality of cells and a cannula extending from a distal end of the hub, the method comprising: orienting the injection attachment such that the cannula points downwardly until the plurality of cells settle together proximate the distal end; inserting the cannula into retinal tissue of a subject; and injecting the plurality of cells into the retinal tissue of the subject using the injection attachment. In certain embodiments, the injection attachment is oriented such that the cannula points downwardly for at least 2 minutes. In certain embodiments, the hub includes an engagement end and a delivery end and an inner surface extending between the engagement end and the delivery end. In certain embodiments, the inner surface tapers inward between the engagement end to the delivery end. In certain embodiments, the plurality of cells is provided to the hub and injected through the cannula.

Further provided herein is an injection attachment for cell transplantation in an eye, comprising: a hub having a proximal end, a distal end, and an inner surface extending therebetween defining an inner volume; and a cannula coupled to the distal end of the hub in fluid communication with the inner volume. In certain embodiments, the inner volume contains a prefilled volume of stem cells. In certain embodiments, the inner volume is a generally funnel shape. In certain embodiments, at least 90% the cells settle proximate the distal end of the hub when the distal end of the hub points downward. In certain embodiments, the hub comprises: a body defining the proximal end of the hub; and a bushing coupled to an inner surface of the body and defining the distal end of the hub. In certain embodiments, the inner surface tapers inward from the proximal end to the distal end such that a diameter of the inner surface is smaller at the distal end than the diameter of the inner surface at the proximal end. In certain embodiments, the inner volume does not retain stem cells after an injection.

Other objects, features and advantages of the present disclosure will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: A side view of injection attachment 100.

FIG. 2: A partial section view of injection attachment 100.

FIG. 3: Flow diagram of rodent and clinical/NHP formulation.

FIG. 4: The dosing mechanism 200 (e.g., CONSTELLATION® Vision System) is attached to tubing from the Viscous Fluid Control Pack 210, MICRODOSE™ Injection Kit 220, and injection attachment 100. At times, a manual syringe (i.e., plunger manipulated by hand) and different cannula tips and cannulas may be used.

FIGS. 5A-5C: For injection attachment 100, gauge does not impact cell aggregate integrity. (FIG. 5A) Two users loaded iPRP cell aggregates and ejected them through either a 31 or 33G POLYTIP® Cannula to determine if there were any detrimental effects to the aggregate product when passing through a smaller cannula. Neither % Volume nor Average Diameter in the gate were appreciably impacted by cannula gauge. The % biomass volume over the 17.03 diameter is calculated by obtaining the biomass over the 17.03 diameter and dividing it by the total biomass (left/primary y-axis). (FIG. 5B) Comparison of median diameter of the iPRP aggregates after either no injection, injection through the injection attachment 100 using the dosing mechanism 200 (e.g., Constellation) to increase pressure to 10 PSI, hold, then increase to 16 PSI and release, or injection at 16 PSI. (FIG. 5C) Comparison of percent aggregate biomass of the iPRP aggregates after either no injection, injection through the custom cannula using the dosing mechanism 200 (e.g., Constellation) to increase pressure to 10 PSI, hold, then increase to 16 PSI and release, or injection at 16 PSI.

FIG. 6: iPRP aggregates can be maintained in suspension to ensure consistent dose delivery. Injections 1-34 show the percent of expected cell recovery following the standard FCDI bench testing dose injection protocol. The low recovered dose in surgeon's orientation (Injection #35), was likely due to settling of the dose in the syringe due to the time and angle that the syringe was being held. Trial #36 (5 minute rolled dose) shows that the low recovery from surgeon's orientation was not simply due to the time the dose was held in the syringe. The percent of cells expected is shown. For example, if 1M cells were recovered but a 1M dose was the intended target, it would be 100% of expected, but if a 2M dose was the intended target and only 1M cells were recovered, the recovery would be 50% of expected.

FIG. 7: Flicking a syringe loaded with photoreceptor cell aggregates can redistribute settled aggregates and deliver an appropriate dose with standard cannula. Cell aggregates can fall out of the formulation with standard cannulas, thus, it was attempted to identify methods to maintain proper aggregate distribution in the syringe. Flicking, vortexing or no mixing with the standard cannula are shown in FIG. 7. Following dose generation, standard syringes were prepared to inject 2 million cells per 50 μL (NHP dose). Syringes were then maintained for 5 minutes in a horizontal position after which doses were mixed or not according to the test condition. A clear lack of recovered cells from the no mix condition can be seen, while recoveries around the targeted 2 million cells were obtained in the vortexed and flicked conditions. Thus, without effective mixing with the standard cannula, sufficient dosing is not achieved and variability in surgical injections remain.

FIGS. 8A-8F: Mock injections with the injection attachment 100 and aggregate settling deliver a more consistent dose than the suspension method. (FIG. 8A) Letting iPRP aggregates settle in the off-the-shelf cannula (MedOne #3262) prevented proper delivery of the dose. (FIG. 8B) Technical drawing of the off-the-shelf 33G POLYTIP® Cannula (MedOne #3262). (FIG. 8C) Image of aggregates (white ring) trapped in the hub of the off-the-shelf cannula after the plunger had been fully depressed. Arrows in B and C show cannula hub 301 where aggregates become trapped. (FIG. 8D) Technical drawing of funnel shaped hub 110 of, injection attachment 100. (FIG. 8E and 8F) Two experiments comparing the injection attachment 100 and the off-the-shelf cannula demonstrate that the settling method delivers a more consistent dose than the suspension method.

FIG. 9: Passing iPRP aggregates through a 33G cannula twice does not appreciably impact the aggregate size compared to a single pass. Two users ejected photoreceptor cell aggregates through a standard 33G POLYTIP® cannula after the dose had been loaded into the syringe through an 18G needle (single pass) or through a 33G cannula (double pass). These aggregates were then analyzed on a Multisizer to determine if there was any impact on aggregate integrity. Neither % Volume nor Average Diameter in the gate were appreciably impacted by the number of times aggregates passed through the cannula.

FIG. 10: Schematic of dose preparation for concentration studies. After the iPRP aggregate cells were thawed and washed, they were resuspended at a concentration higher than the target concentration. A sample from this Bulk was taken for cell counts. Once the concentration of the Bulk was determined, it was distributed to separate tubes, and enough vehicle was added to dilute the Master Doses to their final concentrations. These Master Doses were then dispensed into aliquots intended for injection.

FIG. 11: Master dose variability from concentration studies. The percent of expected recovery from 28 different master doses of iPRP aggregates across 10 experiments averages 93% with a coefficient of variation of 11.4% with the injection attachment 100.

FIG. 12: Schematic of final dose preparation. After the iPRP aggregate cells were thawed and washed, they were resuspended at a concentration higher than the target concentration. A sample was taken for cell counts, the bulk volume (minus 20 μL) was moved to a separate tube and enough vehicle was added to dilute the Master Dose to the final concentration. This Master Dose was then dispensed into aliquots intended for injection.

FIG. 13: Master dose variability generated from clinical workflow. Master Doses of iPRP aggregates made with the clinical workflow (1 Bulk to 1 Master Dose) average 93.5% of the expected cell concentration with a coefficient of variation of 5.0%.

FIG. 14: Dose-to-dose coefficient of variation of dose for administration ranged from 7.5% to 15.5%. In 8 experiments with the injection attachment 100 where the Master Dose was generated from a single Bulk preparation of iPRP aggregates, 5 and 12 aliquots ranging in volume from 60 to 210 μL were sampled and dissociated for counts to calculate the % of Expected Recovery and determine dose-to-dose variability. All doses were within±55% of their expected cell concentration.

FIGS. 15A-15C: Dose recoveries for varying cell concentrations. After observing that injected cell recovery was lower than expected, calculated doses were interrogated over a range of concentrations with the injection attachment 100. (FIG. 15A) After 82 calculated doses were prepared over 11 experiments and 6 lots, it was determined that in order to inject 1 million cells (clinical dose), a 1.7 million cell dose was needed, and in order to inject 2 million cells (NHP dose), a 2.8 million cell dose needs to be prepared. (FIG. 15B) For the 1.7 million cell dose, in 6 experiments, across 3 lots and 25 injections, the average delivered injection contained 1.04 million cells, with a coefficient of variation of 19%. 84% ( 21/25) of the doses fell within±30% of the one million cell target. (FIG. 15C) For the 2.8 million cell dose, in 3 experiments, across 3 lots and 15 injections, the average delivered injection contained 2.1 million cells, with a coefficient of variation of 16%. 93% ( 14/15) of the doses fell within±30% of the two million cell target. For the three and four million cell doses, a linear fit line was extrapolated across all data points generated for 391 individual injections across 2 iPSC lines, 8 iPRP lots (16 sublots) for all four doses, a best fit equation of y=0.0348x−0.1239. Using this equation, it was calculated that doses of 4.5 and 5.95 million cells loaded were needed to achieve the target doses of three and four million cells injected, respectively.

FIGS. 16A-16C: Immunofluorescence images demonstrating transplanted cell survival and photoreceptor identity in an injection in the eye superior to the fovea. (FIGS. 16A-16C) Representative micrographs of transplanted cell area (multiple areas imaged). Stem121 labels the cytoplasm of all transplanted human cells (note that the host RPE is also highly autofluorescent). AIPL1 labels rod and cone photoreceptors. There is some cross-reaction of AIPL1 with host NHP photoreceptors, however the labeling of transplanted human photoreceptors is much brighter. In a normal retina with an intact ONL and outer limiting membrane, iPRP cell aggregates primarily reside in the subretinal space and do not integrate within the host ONL, as expected. A′-C′) Corresponding brightfield (DIC images). Transplanted cells were often contacted by pigmented RPE (brownish cells surrounding AIPL1/Stem121 staining). It was unclear if this was an artifact of thick cryosectioning, or was a biological response to the xenograft. Note that separation of the neural retina is an artifact of tissue processing and sectioning. T=transplanted cell region. ONL=outer nuclear layer. INL=Inner nuclear layer.

FIG. 17A: A side cross sectional view of the injection attachment 100 of FIG. 1 couled to a syringe, including a settled volume of cells in the distal end of the funnel shaped hub 110.

FIG. 17B: A side cross sectional view of the injection attachment 100 of FIG. 1 couled to a syringe, including a settled volume of cells in the distal end of the funnel shaped hub and inserted into the eye of a subject 110.

### DETAILED DESCRIPTION

### I. Description of Illustrative Embodiments

There is an unmet need for improved methods and devices for the delivery of cell compositions for the reliable delivery of precise high-density doses to the target tissue without damage to or loss of cells. The integrity of the cell composition can be negatively impacted by how it is handled, such as with multiple handling steps of changing needles. Cell compositions may comprise single cell suspensions or cell aggregates and be unicellular or multicellular. The cells in the composition may settle out of the formulation buffer which can impact dose formulation and problems during dose delivery. In particular, standard cannulas cannot be used to reliably provide delivery of precise doses as cells in the composition may be trapped in the cannula hub due to settling and improper resuspension techniques. Thus, it was hypothesized that a custom cannula in which the hub is shaped like a funnel would improve injection dose consistency as there would be essentially no space for the cell composition, such as single cells or aggregates, to become trapped.

In some aspects, the cell aggregates may comprise a median aggregate diameter of about 40-60 um, such as 40-45, 45-55, 50-55, or 50-60 um. The aggregate integrity may be determined by measuring the aggregate diameter and % volume>17.03 um. In some aspects, the aggregate size may be between 10-200 um, such as between 15-170 or 17-168 um.

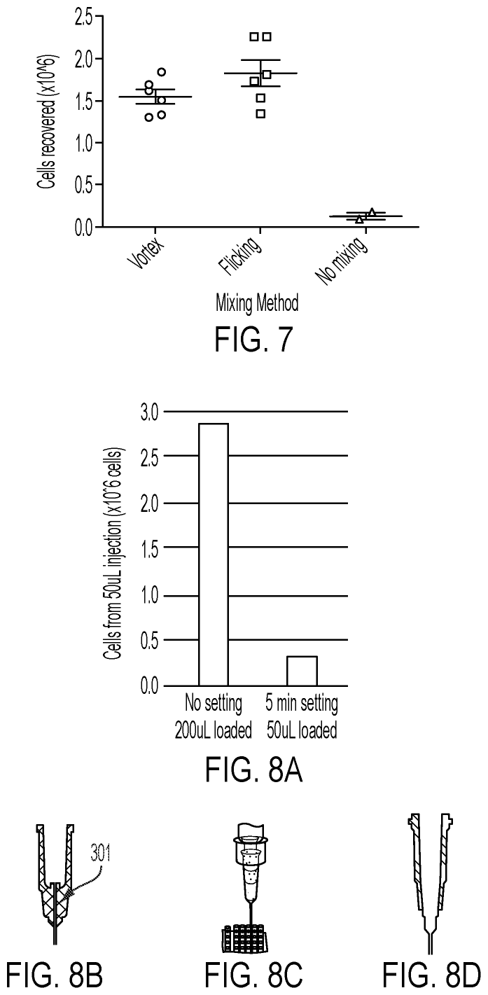

With the knowledge that a cell composition, such as cell aggregates, could fall out of the formulation buffer once loaded into the syringe and therefore result in an inaccurate dose to be injected, it was attempted to identify methods to maintain proper aggregate distribution in the syringe. To prevent aggregate settling, flicking and vortexing were tested and shown (FIG. 7) to be effective for mixing with the standard cannula. In this ‘resuspension’ method, an excessive volume is pulled into a syringe attached to a cannula, air bubbles are removed by flicking and ejecting out the volume that is not needed, and then remaining volume is injected. The issue with this method, is that the cells, such as aggregates, can settle such as they are not equally distributed throughout the volume. Thus, the resuspension method can result in a non-uniform cell suspension that leads to over or underdose. Further, even with flicking or vortexing, the loading and injection needs to be performed quickly and, even then, variability in cell compositions delivered during surgical injections remains.

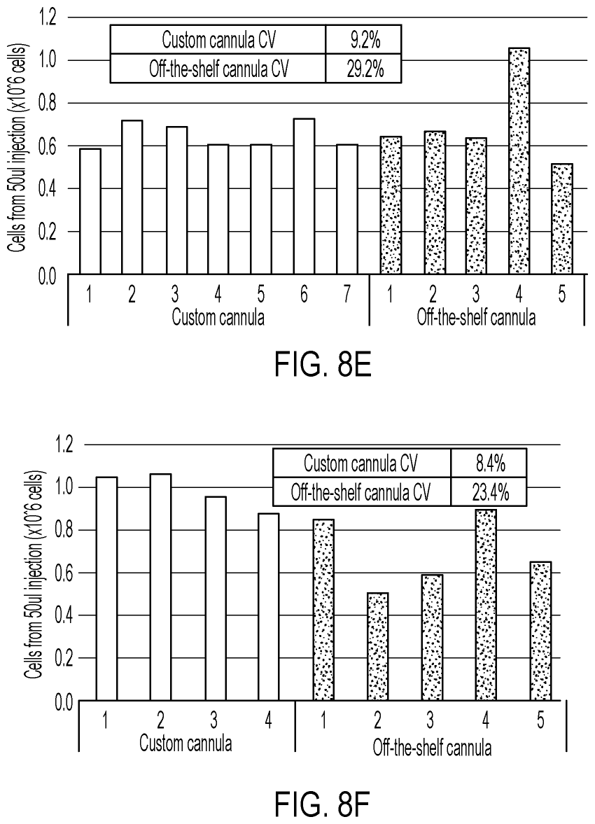

For this reason, it was investigated whether purposefully allowing aggregates to settle would allow for injection of a more consistent dose. However, studies with the standard cannula showed that the syringe in which cells were allowed to settle injected less than 25% of the expected dose. It was found that the standard syringe (FIG. 8B) comprises a cannula hub 301 which can trap cells and prevent them being injected, thus, resulting in a lower dose being administered.

Because allowing aggregates to settle toward the cannula tip failed to work initially due to the geometry of the cannula hub, an injection attachment 100, as referred to herein, and as was referred to as the cannula or custom cannula design in the application to which this disclosure claims priority, which uses a new hub—that acted more like a funnel—and a cannula extending distally from said hub, provides a much more consistent dose. The present cannula may comprise a continuous funnel-like flow pattern which reduces drag and turbulence as a liquid passes through the cannula. With this newly designed cannula, a lower dose (e.g., 50 ul) could be loaded through the cannula, allowed to settle toward its tip for 5 minutes, and then injected, eliminating the need to keep aggregates in suspension prior to injection. It was also shown that the settling method with the injection attachment 100 decreased variability in administered doses. Specifically, it was shown that the injection attachment 100 shape and settling method allows for increased precision. With the increased precision, the dead volume can be overcome by applying a compensation factor in order to accurately administer the dose (i.e., the correct number of cells). The percent improvement is shown in FIG. 6, with the doses falling within the 40-90% of the expected range as compared to the surgeon's orientation dose with a recovery of less than 20%. Thus, the present cannula and methods can allow for the delivery of a complete dose of the cell composition.

In particular aspects, the present ‘settling’ method prevents cells from being trapped in the cannula, thereby providing the target dose with accuracy. Specifically, in the present settling method, the dose is flicked to resuspend the cells, such as aggregates, and then pulled into a previously primed syringe attached to a cannula, therefore going into the device through the same cannula that will be used to transplant the cells back out into the injection site, such as the subretinal space. In specific aspects, the syringe attached to the cannula with the loaded dose is the held vertically for about 2-15 minutes to allow the cell, such as cell aggregates, to settle before injecting the dose.

Thus, in certain embodiments, provided herein is an apparatus and method of using the apparatus that enable reliable delivery of cell composition by providing an injection attachment 100 with a funnel shaped hub 110. In particular aspects, the present disclosure provides a cell delivery apparatus that allows for reliable dose delivery for cell compositions (e.g., compositions of single cells or aggregated cells). In certain aspects, the present injection attachment 100 allows for increased precision of dose delivery and accuracy is further improved by implementing a dose compensation factor. Accordingly, in certain embodiments, there are provided herein methods for the delivery of a cell therapy, such as an aggregate iPSC-derived PRP cell therapy.

In addition to improved dose consistency, in the present methods, the injection attachment 100 described herein allows for reduction of dead volume of a cell solution relative to previous methods. For example, the injection attachment 100 as described herein can allowed for aspiration of 50 ul of cell solution for injection into the cannula to deliver a dose of 50 ul, whereas previous cannulas would require aspiration of 200 μL of cell solution for injection to deliver a dose of 50 ul.

Further, in certain aspects, the present injection attachment 100 comprises a smaller diameter (i.e., larger gauge) as compared to the standard cannula. Specially, previous devices have comprised a larger diameter (e.g., 31G) cannula. The larger cannula has been used to decrease shear forces during loading and injection. However, the present studies showed that a smaller diameter cannula (e.g., 33G) does not negatively impact the cell composition, such as cell aggregate composition, contrary to previous expectations that a smaller diameter would cause cell damage or loss. Specifically, it was shown that initial shear force on the cell composition does not affect the cell composition, particularly with the funnel shaped hub of the present injection attachment 100. In addition, the smaller diameter cannula provides the advantage of minimizing the amount of damage at the injection site and a better surgical outcome with less reflux of the cell composition.

In particular aspects, the present hub has essentially no catch points that could increase shear or turbulence as the cells move through the hub. The hub is mostly smooth as the cells transit through the hub and into the cannula shaft. The reduction in turbulent shear stress helps improve cell viability while catch points cause cell waste and damage. Thus, the structure of the present injection attachment 100 allows a reduction in cell loss.

Thus, in some aspects, the present apparatus comprises injection attachment 100 that is coupled to a dosing mechanism 200, such as the CONSTELLATION®, STELLARIS®, and EVA® Vision Systems, or a manual syringe. The injection attachment 100 comprises a funnel shaped hub 110 that prevents any settling or trapping of the cells in the hub which allows for a higher dose of cells to be delivered to the subject. The cannula tip 121 may be a 33 gauge or 31 gauge cannula tip. The present methods also allow for a smaller dose volume to be used, such as less than 200 μL, less than 175 μL, less than 150 μL, less 100 μL, less than 75 μL, less than 50 μL, less than 25 μL, or less than 10 μL. For example, the hub may comprise an inner volume 117 of less than 200 μL, less than 175 μL, less than 150 μL, less 100 μL, less than 75 μL, less than 50 μL, less than 25 μL, or less than 10 μL. The hub may comprise an inner volume of about 10-25 μL, 25-50 μL, 50-75 μL, 75-100 μL, 100-150 μL, 150-175 μL, 175-200 μL, or 200-250 μL. In some aspects, the hub 110 may comprise an inner volume 117 greater than 5 μL, 10 μL, 25 μL, 50 μL, 75 μL, 100 μL, 150 μL, 175 μL, or 200 μL.

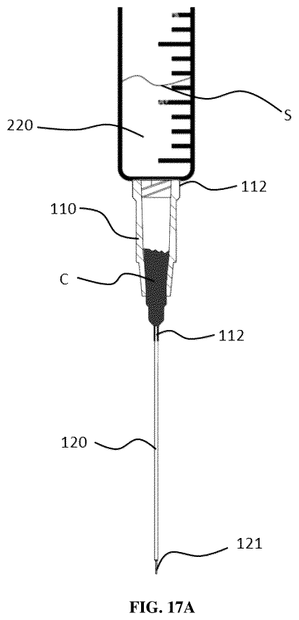

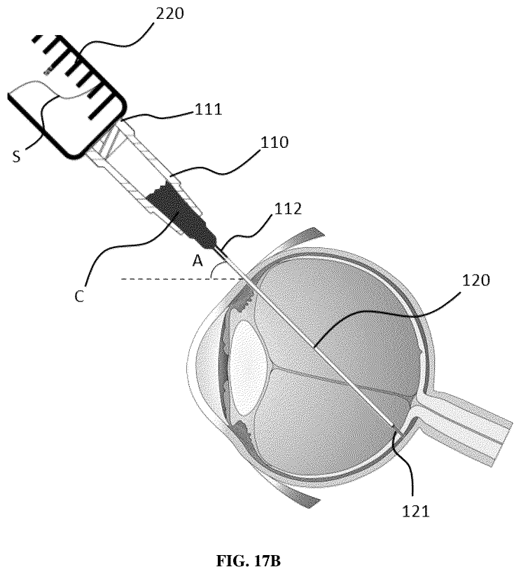

The cell composition may comprise a variety of excipients (e.g., buffers, salts, polymers, proteins, and preservatives), such as to stabilize the cells or to provide physiological osmolality. The cells may be suspended in a formulation buffer. The buffer may be a phosphate, citrate, acetate or other organic acid buffer. The buffer may comprise DMSO, benzonase, or albumin. In particular aspects, the buffer is a balanced salt solution (BSS), such as BSS comprising DMSO or albumin, particular human serum albumin (HSA), such as 0. 1%, 0.2%, 0.3%, 0.4%, or 0.5% HSA buffer. The cells may be resuspended prior to loading, such as by vortexing or manually flicking the vial comprising a composition of cells. In some aspects, as shown in FIG. 17A, the cells, such as the cell aggregates, are allowed to settle in the cannula after loading and prior to injection, such as 1, 2, 3, 4, 5, 6, 7, 8, 9, or 10 minutes, particularly 5 minutes. In certain aspects, the cells in the composition are allowed to settle in the cannula after loading and prior to injection, such as more than 1, 2, 3, 4, 5, 6, 7, 8, 9, or 10 minutes. In certain aspects, the cells in the composition are allowed to settle in the cannula after loading and prior to injection, such as less than 1, 2, 3, 4, 5, 6, 7, 8, 9, or 10 minutes. In certain aspects, the cells in the composition are allowed to settle in the cannula after loading and prior to injection, such as for about 1-3, 2-5, 3-6, 4-7, 5-8, 6-9, or 7-10 minutes. In certain aspects, the cells in the composition are allowed to settle in the cannula after loading and prior to injection, such as for at least 1, 2, 3, 4, 5, 6, 7, 8, 9, or 10 minutes. In certain aspects, the cells in the composition are allowed to settle in the cannula after loading and prior to injection, such as for at least 1-3, 2-5, 3-6, 4-7, 5-8, 6-9, or 7-10 minutes.

Previously, a single-pass method has been used which comprises loading the cell composition through a first larger needle, such as a 18G needle, and then replacing the larger diameter needle with a smaller diameter needle, such as a 31G cannula, prior to injection. Generally, a larger diameter needle is used for loading to decrease shear forces during loading and a smaller diameter needle is used for injections to minimize the amount of damage or pain at the site of injection. In the single-pass method, a larger dose (e.g., 200 μL) is loaded into the syringe to help aid in the removal of air in the syringe and cannula. Once the air is removed, the volume is then dispensed until a lower (e.g., 50 μL) dose remains. The single-pass method requires changing of the needle which can result in loss and damage to integrity of the cell composition as well as variability in doses administered.

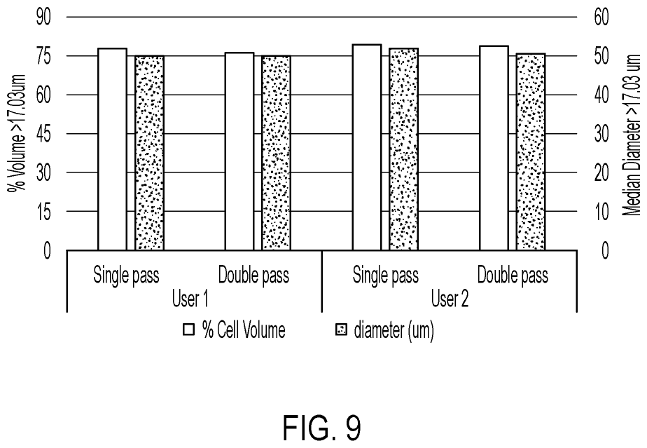

However, in certain embodiments, the present methods are directed to a dual-pass method in which the needle is not changed between loading and injection. The present studies showed that aggregates could pass through the cannula twice without consequential damage (FIG. 9). This dual pass method also allowed for an overhaul of the syringe handling prior to dose delivery. In this dual-pass method, the cells are loaded and ejected through the same cannula which decreases the handling steps as well as decreasing the chance of contamination and needle sticks. In particular, in the dual-aspect method, the same volume dose (e.g., 50 μL) may be loaded and injected through the single needle as compared to the initial large volume used in the single-pass method.

Thus, in certain embodiments, there is provided herein a delivery apparatus and delivery methods for the delivery of cell therapies, such as PRP cell aggregates. The present methods allow for a consistent and high dose of cells to be delivered without the loss of large of amounts of cells and in a lower dose volume. In specific aspects, the cell therapy may be delivered subretinally to an eye of a subject by the present apparatus comprising a cannula with a funnel shaped hub. In some aspects, a bleb is inserted in the retina prior to administering the cells.

### II. Definitions

The term “purified” does not require absolute purity; rather, it is intended as a relative term. Thus, a purified population of cells is greater than about 90% 91%, 92%, 93%, 94%, 95%, 96%, 97%, 98%, 99% or 100% pure, or, most preferably, essentially free of other cell types.

As used herein in the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “essentially” is to be understood that methods or compositions include only the specified steps or materials and those that do not materially affect the basic and novel characteristics of those methods and compositions.

As used herein, a composition or media that is “substantially free” of a specified substance or material contains≤30%, ≤20%, ≤15%, more preferably≤10%, even more preferably≤5%, or most preferably≤1% of the substance or material.

The terms “substantially” or “approximately” as used herein may be applied to modify any quantitative comparison, value, measurement, or other representation that could permissibly vary without resulting in a change in the basic function to which it is related.

The term “about” means, in general, within a standard deviation of the stated value as determined using a standard analytical technique for measuring the stated value. The terms can also be used by referring to plus or minus 5% of the stated value.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

The term “stem cell” refers herein to a cell that under suitable conditions is capable of differentiating into a diverse range of specialized cell types, while under other suitable conditions is capable of self-renewing and remaining in an essentially undifferentiated pluripotent state. The term “stem cell” also encompasses a pluripotent cell, multipotent cell, precursor cell and progenitor cell. Exemplary human stem cells can be obtained from hematopoietic or mesenchymal stem cells obtained from bone marrow tissue, embryonic stem cells obtained from embryonic tissue, or embryonic germ cells obtained from genital tissue of a fetus. Exemplary pluripotent stem cells can also be produced from somatic cells by reprogramming them to a pluripotent state by the expression of certain transcription factors associated with pluripotency; these cells are called “induced pluripotent stem cells” or “iPSCs”.

The term “pluripotent” refers to the property of a cell to differentiate into all other cell types in an organism, with the exception of extraembryonic, or placental, cells. Pluripotent stem cells are capable of differentiating to cell types of all three germ layers (e.g., ectodermal, mesodermal, and endodermal cell types) even after prolonged culture. A pluripotent stem cell is an embryonic stem cell derived from the inner cell mass of a blastocyst. In other embodiments, the pluripotent stem cell is an induced pluripotent stem cell derived by reprogramming somatic cells.

The term “differentiation” refers to the process by which an unspecialized cell becomes a more specialized type with changes in structural and/or functional properties. The mature cell typically has altered cellular structure and tissue-specific proteins.

As used herein, “undifferentiated” refers to cells that display characteristic markers and morphological characteristics of undifferentiated cells that clearly distinguish them from terminally differentiated cells of embryo or adult origin.

“Embryoid bodies (EBs)” are aggregates of pluripotent stem cells that can undergo differentiation into cells of the endoderm, mesoderm, and ectoderm germ layers. The spheroid structures form when pluripotent stem cells are allowed to aggregate under non-adherent culture conditions and thus form EBs in suspension.

An “isolated” cell has been substantially separated or purified from others cells in an organism or culture. Isolated cells can be, for example, at least 99%, at least 98% pure, at least 95% pure or at least 90% pure.

An “embryo” refers to a cellular mass obtained by one or more divisions of a zygote or an activated oocyte with an artificially reprogrammed nucleus.

An “embryonic stem (ES) cell” is an undifferentiated pluripotent cell which is obtained from an embryo in an early stage, such as the inner cell mass at the blastocyst stage, or produced by artificial means (e.g. nuclear transfer) and can give rise to any differentiated cell type in an embryo or an adult, including germ cells (e.g. sperm and eggs).

“Induced pluripotent stem cells (iPSCs)” are cells generated by reprogramming a somatic cell by expressing or inducing expression of a combination of factors (herein referred to as reprogramming factors). iPSCs can be generated using fetal, postnatal, newborn, juvenile, or adult somatic cells. In certain embodiments, factors that can be used to reprogram somatic cells to pluripotent stem cells include, for example, Oct4 (sometimes referred to as Oct 3/4), Sox2, c-Myc, and Klf4, Nanog, and Lin28. In some embodiments, somatic cells are reprogrammed by expressing at least two reprogramming factors, at least three reprogramming factors, or four reprogramming factors to reprogram a somatic cell to a pluripotent stem cell.

An “allele” refers to one of two or more forms of a gene. Diploid organisms such as humans contain two copies of each chromosome, and thus carry one allele on each.

The term “homozygous” is defined as containing two of the same alleles at a particular locus. The term “heterozygous” refers to as containing two different alleles at a particular locus.

A “haplotype” refers to a combination of alleles at multiple loci along a single chromosome. A haplotype can be based upon a set of single-nucleotide polymorphisms (SNPs) on a single chromosome and/or the alleles in the major histocompatibility complex.

As used herein, the term “haplotype-matched” is defined as the cell (e.g. iPS cell) and the subject being treated share one or more major histocompatibility locus haplotypes. The haplotype of the subject can be readily determined using assays well known in the art. The haplotype-matched iPS cell can be autologous or allogeneic. The autologous cells which are grown in tissue culture and differentiated to PRP cells inherently are haplotype-matched to the subject.

“Substantially the same HLA type” indicates that the Human Leukocyte Antigen (HLA) type of donor matches with that of a patient to the extent that the transplanted cells, which have been obtained by inducing differentiation of iPSCs derived from the donor's somatic cells, can be engrafted when they are transplanted to the patient.

“Super donors” are referred to herein as individuals that are homozygous for certain MHC class I and II genes. These homozygous individuals can serve as super donors and their cells, including tissues and other materials comprising their cells, can be transplanted in individuals that are either homozygous or heterozygous for that haplotype. The super donor can be homozygous for the HLA-A, HLA-B, HLA-C, HLA-DR, HLA-DP or HLA-DQ locus/loci alleles, respectively.

“Feeder-free” or “feeder-independent” is used herein to refer to a culture supplemented with cytokines and growth factors (e.g., TGFβ, bFGF, LIF) as a replacement for the feeder cell layer. Thus, “feeder-free” or feeder-independent culture systems and media may be used to culture and maintain pluripotent cells in an undifferentiated and proliferative state. In some cases, feeder-free cultures utilize an animal-based matrix (e.g. MATRIGEL™) or are grown on a substrate such as fibronectin, collagen, or vitronectin. These approaches allow human stem cells to remain in an essentially undifferentiated state without the need for mouse fibroblast “feeder layers.”

“Feeder layers” are defined herein as a coating layer of cells such as on the bottom of a culture dish. The feeder cells can release nutrients into the culture medium and provide a surface to which other cells, such as pluripotent stem cells, can attach.

The term “defined” or “fully-defined,” when used in relation to a medium, an extracellular matrix, or a culture condition, refers to a medium, an extracellular matrix, or a culture condition in which the chemical composition and amounts of approximately all the components are known. For example, a defined medium does not contain undefined factors such as in fetal bovine serum, bovine serum albumin or human serum albumin. Generally, a defined medium comprises a basal media (e.g., Dulbecco's Modified Eagle's Medium (DMEM), F12, or Roswell Park Memorial Institute Medium (RPMI) 1640, containing amino acids, vitamins, inorganic salts, buffers, antioxidants, and energy sources) which is supplemented with recombinant albumin, chemically defined lipids, and recombinant insulin. An example of a fully defined medium is Essential 8™ medium.

For a medium, extracellular matrix, or culture system used with human cells, the term “Xeno-Free (XF)” refers to a condition in which the materials used are not of non-human animal-origin.

“Pre-confluent” refers to a cell culture in which the proportion of the culture surface which is covered by cells is about 60-80%. Usually, pre-confluent refers to a culture in which about 70% of the culture surface is covered by cells.

The term “neural retinal progenitors” or “NRPs” refers to cells which are restricted in their differentiation potential to neural retina cell types.

The term “photoreceptor” or “PR” cells refer to cells that are within the photoreceptor lineage (i.e., maturation) pathway, both before and after upregulation of expression of rhodopsin (rods) or any of the three cone opsins (cones), which encompasses both early and late markers of photoreceptor cells (rod, cone or both).

The terms “photoreceptor precursor cells” or “PRP” refer to cells differentiated from embryonic stem cells or induced pluripotent stem cells which can differentiate into photoreceptor cells that expresses the cell marker rhodopsin or any of the three cone opsins. The photoreceptors may be rod and/or cone photoreceptors.

The term “retinal degeneration-related disease” is intended to refer to any disease resulting from innate or postnatal retinal degeneration or abnormalities. Examples of retinal degeneration-related diseases include retinal dysplasia, retinal degeneration, age-related macular degeneration, Stargardt disease, Best disease, choroideremia, inherited macular degeneration, myopic degeneration, RPE tears, macular hole, diabetic retinopathy, retinitis pigmentosa, inherited retinal disease or degeneration, inherited macular degeneration, cone-rod dystrophy, rod-cone dystrophy, congenital retinal dystrophy, Leber congenital amaurosis, retinal detachment, and retinal trauma.

The term “ocular condition” as used herein refers to a disease, condition, ailment or injury of the ocular region. In some embodiments, the ocular condition can include a condition of a posterior segment of the eye. In other embodiments, the ocular condition can include a condition of the anterior segment of the eye. The ocular condition may be related to retinal epithelial cells and/or photoreceptor cells. The ocular condition may be related to or associated with diabetes (e.g., diabetic macular edema, retinal arterial occlusive disease, or diabetic retinopathy), age (e.g., age-related macular degeneration, choroidal neovascularization, subretinal fibrosis, or glaucoma), inflammation (e.g., Behcet's disease, posterior uveitis, serpignous choroiditis, uveitis syndrome, cytomegalovirus retinitis, or endophthalmitis), genetics (e.g., Coat's disease, or familial exudative vitreoretinopathy), or cancer (e.g., posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, retinal cancers, or intraocular lymphoid tumors). The disease may be retinopathy, such as acute and chronic macular neuroretinopathy, or central serous chorioretinopathy. For example, the ocular condition can include, but not limited to, age-related macular degeneration, choroidal neovascularization, diabetic macular edema, acute and chronic macular neuroretinopathy, central serous chorioretinopathy, macular edema, acute multifocal placoid pigment epitheliopathy, Behcet's disease, birdshot retinochoroidopathy, posterior uveitis, posterior scleritis, serpignous choroiditis, subretinal fibrosis, uveitis syndrome, Vogt-Koyanagi-Harada syndrome, retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, carotid artery disease (CAD), frosted branch angitis, sickle cell retinopathy, angioid streaks, familial exudative vitreoretinopathy, Eales disease, proliferative vitreal retinopathy, diabetic retinopathy, retinal disease associated with tumors, congenital hypertrophy of the retinal pigment epithelium (RPE), posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors, myopic retinal degeneration, acute retinal pigment epithelitis, glaucoma, endophthalmitis, cytomegalovirus retinitis, retinal cancers, and any combinations thereof. Injuries to the eye may include bruises, punctures, scratches, penetrating injury, perforation injury, or injury due to an intraocular foreign body.

A “therapeutically effective amount” used herein refers to the amount of a compound that, when administered to a subject for treatment of a disease or condition, is sufficient to affect such treatment.

“Inducer” is defined herein as a molecule that regulates gene expression such as activating genes within a cell. An inducer can bind to repressors or activators. Inducers functions by disabling repressors.

As used herein, the term “engrafted” bilayer refers to engraftment of the transplanted cells into the host retina and forming pre-synaptic and post-synaptic machinery such that the transplanted and host cells are poised to form a synapse.

As used herein, the term “biodegradable” refers to a material that provides initial structural support to delivered cells, but degrades over time into products that are not toxic to the transplant host and do not contribute to donor site morbidity.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate, particularly a human Non-limiting examples of human patients are adults, juveniles, infants and fetuses.

As used herein, “smooth” refers to the cannula hub having essentially no protrusions projecting inwardly from the inner hub surface.

### III. Cell Delivery Apparatus

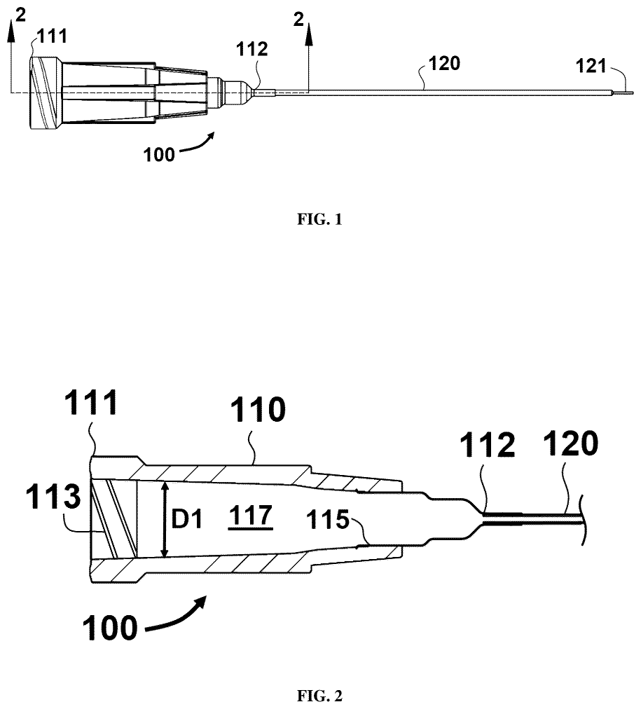

In some aspects, the present disclosure provides an apparatus for the delivery of a composition of cells. The delivery device may comprise an injection attachment with a cannula and a funnel shaped hub, such as injection attachment 100 in FIGS. 1-2. The cannula at distal end 112 of the hub 110 in FIG. 1 may have an outer diameter of less than 0.3 mm, such as a range of 0.15 mm to 0.3 mm, 0.20 mm to 0.25 mm, 0.25 mm to 0.3 mm, or 0.15 mm to 0.25 mm. The cannula hub 110, such as at distal end 112 in FIG. 1, may be a 22 gauge, 23 gauge, 24 gauge, 25 gauge, 26 gauge, 27 gauge, or 28 gauge cannula hub. For example, a micro-cannula may be used. The injection attachment 100 may have a cannula shaft, such as a 25 gauge cannula (e.g., cannula 120 in FIG. 1). At the distal end of cannula 120, the shaft tapers to a smaller cannula tip 121 attached to the cannula 120. A cannula 120 may be provided with a tapered design having a small gauge tip (e.g., 121 in FIG. 1), such as an outer diameter in the range of about 0.30 mm to 0.18 mm or smaller. The cannula tip 121 may have an outer diameter of approximately 0.1 mm, 0.12 mm, 0.14 mm, 0.16 mm, 0.18 mm, 0.2 mm, 0.22 mm, 0.24 mm, 0.26 mm, 0.28 mm, or 0.3 mm. The cannula tip 121 may be a 30 gauge (e.g., 0.30 mm diameter), 31 gauge, 32 gauge, 33 gauge, 34 gauge (e.g., 0.18 mm diameter), 35, gauge, 36 gauge, 37 gauge, 38 gauge (e.g., 0.12 mm diameter), 39 gauge, or 40 gauge (e.g., 0.10 mm diameter) cannula tip, particularly a cannula tip of 30-34 gauge of about 0.30 mm to about 0.18 mm in diameter. For example, cannula 120 may be a 25 gauge cannula 120 with a 33 gauge cannula tip 121. Additionally, the cannula tip 121 may be made of a flexible polyimide, metal, or other similar material. The tip may comprise a length of 1 mm, 2 mm, 3 mm, 4 mmm, or 5 mm and may be rigid or flexible as well as extendable.

The injection attachment 100 may be attached to a syringe 220, such as a syringe that allows controlled microinjection, such as a MICRODOSE™ injector. The injection attachment 100 may comprise a coupling mechanism 113, such as a luer lock hub, or connection to the syringe 220, extension tube 230, or infusion line 240. The tubing may be silicone tubing, such as silicone tubing. The device may further comprise a Viscous Fluid Control Pack 210. In other aspects, the tube 230 may be packed with the syringe 220, such as the MICRODOSE™ injector, without a Viscous Fluid Control Pack 210.

Embodiments of the present disclosure include methods of use comprising a cannula. Referring now to FIGS. 1-2, an injection attachment 100 is shown comprising a funnel shaped hub 110 a cannula 120, and a cannula tip 121. FIG. 1 illustrates a side view of injection attachment 100, while FIG. 2 illustrates a partial section view taken along line 2-2 of FIG. 1.

In exemplary embodiments, coupling mechanism 113, such as a luer lock hub, may be coupled to a dosing mechanism 200 (not shown), including for example, a CONSTELLATION® Vision System, or a manual syringe. It is understood that the listed dosing mechanisms are merely exemplary of particular embodiments, and that other embodiments according to the present disclosure may be coupled to any suitable dosing mechanism.

Referring now to FIG. 2, in the embodiment shown, the funnel shaped hub 110 comprises an inner volume 117 defined by a proximal end 111, as referred to herein, and as was referred to as the first end in the application to which this disclosure claims priority, a distal end 112, as referred to herein, and as was referred to as the second end in the application to which this disclosure claims priority, and an inner surface 115. The term “funnel shaped” as referred to herein may be broadly defined as an inner volume 117 configured to direct particles (e.g., cells suspended in fluid) through the hub 110 into the cannula 120 while minimizing or eliminating the presence of retained particles. In specific embodiments, proximal end 111 may comprise a threaded portion (e.g., coupling mechanism 113) or other suitable coupling mechanism (including, for example, a “Luer-Lock” type design) configured to engage the dosing mechanism. Coupling mechanism 113 may include an interior surface configured to couple to an exterior surface of the dosing mechanism 200. The coupling mechanism 113 may include, for example, angled protrusions that are received by corresponding protrusions on the dosing mechanism 200 to couple by threaded engagement. The coupling mechanism 113 and dosing mechanism 200 may be coupled by snap-fit, glue, epoxy, weld, or other engagements sufficient to prevent unintended detachment of the coupling mechanism 113 and dosing mechanism 200. During use, the dosing mechanism can transfer contents from inner volume 117, through cannula 120 and cannula tip 121, and into a subject, as shown in FIG. 17B.

As shown in FIG. 2, inner surface 115 may be generally funnel shaped. The funnel shape helps minimize retained particles after an injection. The inner surface 115 is continually tapered from proximal end 111 (e.g., an engagement end) to distal end 112 (e.g., a delivery end). The taper may include a constant angle between the proximal end 111 and second end 122. As shown in FIG. 2, the taper may include a variable angle between the proximal end 111 and second end 122. In the example shown, an inner diameter D1 (as measured across inner surface 115 in inner volume 117) is decreasing as measured from proximal end 111 toward distal end 112. Although both sides shown in FIG. 2 mirror each other, it is contemplated that the inner surface 115 can taper toward one side, or may overall taper inward but may taper inward and outward between the proximal end 111 and the distal end 112. In some embodiments, one side of the inner surface 115 may be flat. In this manner, inner surface 115 does not comprise any protrusions or other features that could restrict contents of inner volume 117 from exiting hub 110 and entering cannula 120. Accordingly, contents of inner volume 117 (e.g. a suspension of cells, therapeutic agents, or other suitable contents) can move without restriction from proximal end 111 of injection attachment 100 to distal end 112, and then further into cannula 120 and tip 121. The method and injection attachment 100 may result in at least 99% of cells contained in the injection attachment 100 being injected into the tissue. In some embodiments, at least 99%, 99.1%, 99.2%, 99.3%, 99.4%, 99.5%, 99.6%, 99.7%, 99.8%, 99.9%, or 100% of cells contained in the injection attachment 100 being injected into the tissue. The continuous taper of inner surface 115 and lack of protrusions into inner volume 117 also allows contents of interior volume to be thoroughly mixed prior to administering to a subject.

In particular embodiments, cannula 120, as referred to herein, and as was referred to as the shift in the application to which this disclosure claims priority, may be configured such that the diameter of the shaft does not restrict or limit the dosage of an injection provided by injection attachment 100. In particular embodiments, cannula 120 may be configured as a 25 gauge cannula or other suitable diameter shaft. In particular embodiments, cannula tip 121 may be configured as a 33 gauge (33G) cannula, 31 gauge (31G) cannula, or other suitable diameter tip. The combination of hub 110 (with an inner surface 115 without protrusions or restrictions), and a suitable diameter of cannula 120 therefore allows injection attachment 100 to provide thorough mixing of the contents of inner volume 117 and administration of the contents without restriction in inner volume 117. Accordingly, the contents of inner volume 117 can be administered to a subject in an accurate and repeatable dosage.

### IV. Cell Delivery Methods

In certain aspects, the present disclosure provides methods for cell transplantation, such as cell aggregates or single cell suspension therapies. The present methods may be applied to various cell transplantation procedures known in the art. The transplantation may comprise providing a volume of cells to a cell delivery apparatus (not shown), the apparatus comprising a proximal end and a distal end opposite the proximal end. The apparatus may include an interior volume configured to receive the volume of cells.

The apparatus may include a cannula (e.g., cannula 120) extending from a funnel shaped hub 110. The cannula may comprise a cannula tip 121. The cannula tip 121 may be a 33 gauge cannula tip. In some embodiments, the cannula tip 121 is a 32 gauge cannula tip, a 31 gauge cannula tip, a 30 gauge cannula tip, a 29 gauge cannula tip, a 28 gauge cannula tip, a 27 gauge cannula tip, a 26 gauge cannula tip, a 25 gauge cannula tip, a 24 gauge cannula tip, a 23 gauge cannula tip, or a 22 gauge cannula tip.

The funnel shaped hub 110 being coupled to the distal end of the apparatus and in fluid communication with the internal volume. The funnel shaped hub 110 may include a proximal end 111 and a distal end 112, with an inner surface 115 extending between the proximal end 111 and the distal end 112. The inner surface 115 may be generally cylindrical between the proximal end 111 and the distal end 112.

The inner surface 115 may have a diameter that is smaller at the distal end 112 than the diameter of the interior wall at the proximal end 111. The inner surface 115 may taper between the proximal end 111 and the distal end 112. The cannula (e.g., cannula 120) may be coupled to the proximal end 111 of the funnel shaped hub 110. The cannula 120 is coupled to the distal end 112 using a glue or suitable epoxy. In some embodiments, the cannula 120 is integrally coupled to the distal end 112. The cannula 120 does not extend past the distal end 112 of the funnel shaped hub 110. As shown in FIG. 2, the cannula 120 is coupled to the distal end 112 such that no part of the cannula 120 enters the inner volume 117.

The apparatus may be oriented such that the distal end is below the proximal end relative to a ground surface for a period of time. The period of time may be sufficient to allow the cells to settle in the distal end of the hub 110. Settling may refer to approximately 90% of the cells settling within the hub 110, as shown in FIG. 17A. In some embodiments, settling means at least 50% of the cells, at least 60% of the cells, at least 70% of the cells, at least 80% of the cells, at least 90% of the cells, at least 95% of the cells, or at least 99% of the cells. In some embodiments, settling means between 50% and 90% of the cells, between 55% and 85% of the cells, between 60% and 80% of the cells, or between 65% and 75% of the cells.

The period of time may be sufficient to allow the cells to settle in the distal end 112 of the funnel shaped hub 110. As shown in FIGS. 17A-17B, the cells (C) may settle in the distal end 112 of the funnel shaped hub 110. Also shown in FIGS. 17A-17B is a liquid solution (S) in which the cells C are suspended. The funnel shaped hub 110 may comprise a transparent material to allow visual inspection of the cells settling therein. The period of time may be between 30 seconds and 10 minutes. The period of time may be between 1 minute and 9 minutes, between 2 minutes and 8 minutes, between 3 minutes and 7 minutes, or between 4 minutes and 6 minutes. The period may be at least 30 seconds, at least 1 minute, at least 2 minutes, at least 3 minutes, at least 4 minutes, at least 5 minutes, at least 6 minutes, at least 7 minutes, at least 8 minutes, at least 9 minutes, or at least 10 minutes. In certain embodiments, a centrifugal force may be applied to the hub to reduce the period of time required for the cells to settle proximate the distal end of the hub.

The method may include the step of inserting the cannula into a tissue of a subject. The cannula tip 121 may be blunt ended and configured to enter the tissue through an incision. In some embodiments, the cannula tip 121 may be sharp ended and configured to pierce the tissue to enter. The tissue may be retinal tissue.

As shown in FIG. 17B, the apparatus may be held at an angle (A) while injecting the cells into the tissue of the subject. The apparatus may be held at an angle A of approximately 45 degrees relative to the ground surface during injection. The apparatus may be held at an angle A of approximately 20 degrees, 25 degrees, 30 degrees, 35 degrees, 40 degrees, 45 degrees, 50 degrees, 55 degrees, 60 degrees, 65 degrees, or 70 degrees relative to the ground surface during injection. The apparatus may be held at an angle A between 20 degrees and 70 degrees relative to the ground surface during injection. The apparatus may be held at an angle A between 25 degrees and 65 degrees, between 30 degrees and 60 degrees, between 35 degrees and 55 degrees, between 40 degrees and 50 degrees, or between 45 degrees and 55 degrees relative to the ground surface during injection.

The method may include the step of injecting the volume of cells into the tissue of the subject using the cell delivery apparatus. The volume of cells may be provided to the internal apparatus and injected into the tissue of the subject through the same injection attachment 100. For example, the cannula tip 121, nor the cannula 120, is changed between providing the volume of cells into the internal volume and injecting the cells into the tissue of the subject.

In certain aspects, the present disclosure provides methods for the delivery of cell therapy, such as cell aggregates or single cell suspension therapies. The present methods may be applied to various cell therapies known in the art. The delivery may comprise a microsurgical procedure, such as an ophthalmic procedure, using a microinjection cannula or similar device. The delivery may comprise an injection under the retina by a slow, controlled injection of a cell therapy. The cannula may be coupled to a syringe or system that allows for a pre-defined uniform injection pressure (psi), such as between 0-30 psi (e.g., greater than 2, 4, 6, 8, 10, 12, 14, 16, 18, 20 psi, or a psi of 0-30, 2-28, 4-26, 6-24, 5-20, 15-25, 20-25, or 25-30, or a psi of 0-15, 0-16, 0-17, 0-18, 0-19, or 0-20) which may be controlled by a foot pedal 250 that control the pressure in a variable manner The cells may be stored at an appropriate temperature, such as 4° C.

In certain embodiments, the present delivery device provides a mechanism for delivery to an eye, such as sub-retinal administration, or delivery of a therapeutic medium to a mammalian eye (e.g., a posterior segment of the eye), more particularly a human eye as well as a methodology for treating and/or preventing disorders and/or diseases of the eye, in particular retinal/choroidal disorders or diseases, through such sub-retinal administration of such therapeutic mediums. Such methodologies provide a mechanism for treating a wide array of diseases and/or disorders of an eye of a mammal, more specifically a human eye, and more particularly diseases or disorders involving the posterior segment of the eye such as retinal/choroidal disorders or diseases. Such a treatment/prevention methodology also is useable to treat/prevent a number of vision-threatening disorders or diseases of the eye of a mammal including, but not limited to diseases of the retina, retinal pigment epithelium (RPE) and choroid. Such vision threatening diseases include, for example, ocular neovascularization, ocular inflammation and retinal degenerations. Specific examples of these disease states include diabetic retinopathy, chronic glaucoma, retinal detachment, sickle cell retinopathy, age-related macular degeneration, retinal neovascularization, subretinal neovascularization; rubeosis iritis inflammatory diseases, chronic posterior and pan uveitis, neoplasms, retinoblastoma, pseudoglioma, neovascular glaucoma; neovascularization resulting following a combined vitrectomy and lensectomy, vascular diseases retinal ischemia, choroidal vascular insufficiency, choroidal thrombosis, neovascularization of the optic nerve, diabetic macular edema, cystoid macular edema, macular edema, retinitis pigmentosa, retinal vein occlusion, proliferative vitreoretinopathy, angioid streak, and retinal artery occlusion, and, neovascularization due to penetration of the eye or ocular injury. The present methods also can be used to treat ocular symptoms resulting from diseases or conditions that have both ocular and non-ocular symptoms.

In some embodiments, the cell composition preparation and delivery comprise the steps of thawing and resuspending the cell composition in a vial (e.g., 50 mL conical tube). The cell composition may be formulated in a buffer as described herein. The cell composition may be centrifuged, such as 1, 2, 3, 4, 5, or more times (e.g., in a formulation buffer). For clinical applications, a bulk dose of the cell composition may be prepared in a formulation buffer (e.g., 25-50, 30-75, 40-80, 50-90, or 60-100 μL of buffer). At this stage, the cell composition may be dissociated and counted. Next, a master dose may be prepared to reach the target concentration for the cell composition. Finally, doses may be aliquoted (e.g., 50 μL) to appropriate vials. For an animal study, such as rodents, the cell composition may be dissociated and counted before centrifuging the remaining volume. The master dose maybe prepared by aspirating the buffer, resuspending the pellet with remaining supernatant, measuring the volume, and adding formulation buffer to achieve the target concentration. The doses (e.g., 15 μL) may then be aliquoted.

The present apparatus may be used for transplantation such as cell rescue therapy or whole tissue replacement therapy. Certain embodiments can provide use of the present apparatus to enhance ocular tissue maintenance and repair for any condition in need thereof, including retinal degeneration or significant injury. Retinal degeneration may be associated with age-related macular degeneration (AMD), inherited macular degenerations, Stargardt's macular dystrophy, Best disease, choroideremia, inherited retinal degenerations (including retinitis pigmentosa, cone/rod and rod/cone dystrophies), diabetic retinopathy, retinal vascular disease, damage caused by retinopathy pf prematurity (ROP), viral infection of the eye, and other retinal/ocular diseases or injuries/trauma.

In another aspect, the disclosure provides a method of treatment of an individual in need thereof, comprising transplanting a composition comprising cells, such as PRP cells, to said individual. Said composition may be administered to the eye, such as the subretinal space. Such individuals may have inherited macular or retinal degenerations such as retinitis pigmentosa, cone/rod or rod/cone dystrophy, Stargardt's disease, Best disease, choroideremia, retinal dysplasia, retinal degeneration, diabetic retinopathy, congenital retinal dystrophy, Leber congenital amaurosis, retinal detachment, damage caused by retinopathy of prematurity (ROP), or other retinal trauma or injury.

The apparatus and cells described herein can be used for the manufacture of a medicament to treat a condition in a patient in need thereof. The cells can be previously cryopreserved. In certain aspects, the disclosed photoreceptors are derived from iPSCs, and thus can be used to provide “personalized medicine” for patients with eye diseases. In some embodiments, somatic cells obtained from patients can be genetically engineered to correct the disease-causing mutation and differentiated into PRPs. Alternatively, iPSCs generated from a healthy donor or from HLA homozygous “super-donors” can be used.

Various eye conditions may be treated or prevented by the present methods and compositions. The conditions include retinal diseases or disorders generally associated with retinal dysfunction or degradation, retinal injury, and/or loss of retinal pigment epithelium and/or photoreceptors. Conditions that can be treated include, without limitation, degenerative diseases of the retina, such as Stargardt's macular dystrophy, retinitis pigmentosa, rod/cone and cone/rod dystrophies, macular degeneration (such as age-related macular degeneration, myopic macular degeneration, or other acquired or inherited macular degenerations), retinal damage caused by retinopathy of prematurity (ROP) and diabetic retinopathy. Additional conditions include Lebers congenital amaurosis, hereditary or acquired macular or retinal degenerations, Best disease, retinal detachment, gyrate atrophy, choroideremia, pattern dystrophy, other dystrophies of photoreceptor cells, and retinal damage due to damage caused by any one of photic, laser, inflammatory, infectious, radiation, neovascular or traumatic injury. In certain embodiments, methods are provided for treating or preventing a condition characterized by retinal degeneration, comprising administering to a subject in need thereof an effective amount of a composition comprising the PRPs by the present apparatus.

In some embodiments, the present cell delivery device and methods of use thereof provides a mechanism for sub-retinal administration or delivery of cells to a posterior segment of a mammalian eye, more particularly a human eye as well as a methodology for treating and/or preventing disorders and/or diseases of the eye, in particular retinal/choroidal disorders or diseases, through such sub-retinal administration of such therapeutic mediums. Such methodologies provide a mechanism for treating a wide array of diseases and/or disorders of an eye of a mammal, more specifically a human eye, and more particularly diseases or disorders involving the posterior segment of the eye such as retinal/choroidal disorders or diseases. Such a treatment/prevention methodology also is useable to treat/prevent a number of vision-threatening disorders or diseases of the eye of a mammal including, but not limited to diseases of the retina, retinal pigment epithelium (RPE) and choroid. Such vision threatening diseases include, for example, ocular neovascularization, ocular inflammation and retinal degenerations. Specific examples of these disease states include diabetic retinopathy, chronic glaucoma, retinal detachment, sickle cell retinopathy, age-related macular degeneration, retinal neovascularization, subretinal neovascularization; rubeosis iritis inflammatory diseases, chronic posterior and pan uveitis, neoplasms, retinoblastoma, pseudoglioma, neovascular glaucoma; neovascularization resulting following a combined vitrectomy and lensectomy, vascular diseases retinal ischemia, choroidal vascular insufficiency, choroidal thrombosis, neovascularization of the optic nerve, diabetic macular edema, cystoid macular edema, macular edema, retinitis pigmentosa, retinal vein occlusion, proliferative vitreoretinopathy, angioid streak, and retinal artery occlusion, and, neovascularization due to penetration of the eye or ocular injury. The present method also can be used to treat ocular symptoms resulting from diseases or conditions that have both ocular and non-ocular symptoms.

The present delivery methods may be used for cell transplantation of various cell therapies, such as for the treatment of peripheral arterial disease, myocardial infarct, stroke, Parkinson's disease, Huntington's disease, amyotrophic lateral sclerosis (ALS). The cells may be pluripotent stem cells, stem cells, such as mesenchymal stem cells (MSCs), bone marrow cells or neural progenitors cells (NPCs), cardiac precursor cells or iPSC-derived cells, such as neurons, stromal cells, fibroblasts, endothelial cells, epithelial cells, etc. The present device and methods may be used to transplant cells to any soft tissue, including but not limited to, eye, heart, kidney, liver, tumor, or muscle. The dose of the cells may range from hundreds to millions of cells.

### V. Cell Compositions

The term “cell population”, “cell composition”, “cell therapy composition” or “composition of cells” is used interchangeably herein to refer to a group of cells, typically of a common type. The cell population can be derived from a common progenitor or may comprise more than one cell type. An “enriched” cell population refers to a cell population derived from a starting cell population (e.g., an unfractionated, heterogeneous cell population) that contains a greater percentage of a specific cell type than the percentage of that cell type in the starting population. The cell populations may be enriched for one or more cell types and depleted of one or more cell types.

The cell composition may comprise a single cell suspension or cell aggregates. The composition of cells may be autologous or allogenic. The composition of cells may comprise or be derived from PSCs, such as iPSCs or hESCs. The composition of cells may comprise one cell type (i.e., unicellular) or two or more cell types (i.e., multicellular). In some aspects, the term “cells” as used herein refers to a composition of cells. In some aspects, a cell composition may comprise organoids or be a whole tissue replacement.

In some aspects, the cell aggregates may include a cell cluster. The cell cluster may include at least 2 cells. In some embodiments, the cell cluster includes more than 3, more than 4, more than 5, more than 10, more than 15, more than 20, more than 25, or more than 30 cells. In some embodiments, the cell cluster includes at least 3, at least 4, at least 5, at least 10, at least 15, at least 20, at least 25, or at least 30 cells. A single cell may be approximately 9 μm in diameter. In some embodiments, a single cell is approximately 7 μm, approximately 7.5 μm, approximately 8 μm, approximately 8.5 μm, approximately 9 μm, approximately 9.5 μm, approximately 10 μm, approximately 10.5 μm, or approximately 11 μm. The cell composition may include distributions of cell clusters varying in size therein.

In some aspects, the comprises pluripotent stern cells (PSCs) or cells derived therefrom. PSCs may comprise embryonic stem cells (ESCs) or induced pluripotent stem cells (iPSCs) which have the ability to differentiate into several cell types that can be used in drug testing and also in the study and treatment of diseases. PSCs may be differentiated into any of the 216 cell types found in an adult organism, such as neurons, cardiomyocytes, smooth muscle cells, osteocytes, hepatocytes, keratinocytes, insulin-producing cells, hematopoietic cells, and endothelial cells. In particular aspects, the present PSC-derived cells may comprise one or more of the 216 cell types. In some aspects, the cell composition to be transplanted comprises engineered stem cell-derived tissue or organ made up of multiple cell types. The major cell types used from the endoderm include hepatic cells and insulin-producing cells. The mesodermal progenitors obtained from ESCs and iPSCs includes cardiomyocytes, endothelial cells, and hematopoietic cells. These cell types could be used for treatment of ischemic heart disease, repair of ischemic tissue, and to obtain all types of blood cells, respectively. The cells differentiated into the ectoderm lineage include cells of the epidermis, external sense organs, and central and peripheral nervous system, such as functional neurons that can be used for the treatment of neurodegenerative diseases, such as acute spinal cord injury.

In some aspects, the cell composition may be genetically engineered to knock-out and/or knock-in a gene. This may be performed by various genome-editing approaches, including CRISPR technology, zinc finger nuclease (ZFN) and transcription activator-like effector nuclease (TALEN) technologies.

The cell therapy composition can also include other agents. For example, the composition can include an agent that further protects or stabilizes the cells to be transplanted. In certain embodiments, the composition comprises vitamins, minerals, antioxidants, osmoprotectants, viscosity enhancers, coenzymes, membrane stabilizers, lipids, carbohydrates, hormones, growth factors, anti-inflammatory agents, polynucleotides, proteins, peptides, alcohols, organic acids, small organic molecules and the like.

The cell composition can include pharmaceutically acceptable excipients, including any solvent, dispersion medium, diluent, or other suitable herein for the particular formulation desired. Liquid vehicles, dispersion or suspension aids, surfactants, isotonic agents, thickeners or emulsifiers, preservatives, solid binders, lubricants and the like. Remington's The Science and Practice of Pharmacy, 21^{st} Edition, AR Gennaro (Lippincott, Williams & Wilkins, Baltimore, MD, 2006; incorporated herein by reference) is used to formulate pharmaceutical compositions. Various excipients that are available and known techniques for their preparation are disclosed. Any conventional excipient will produce any undesirable biological effect with the substance or derivative thereof, or interact in a deleterious manner with any other ingredient(s) of the pharmaceutical composition. Thus, unless used otherwise, its use is intended to be within the scope of the present invention.

In some embodiments, the pharmaceutically acceptable excipient is at least 95%, 96%, 97%, 98%, 99%, or 100% pure. In some embodiments, the excipient is approved for human use and veterinary use. In some embodiments, the excipient is approved by the US Food and Drug Administration. In some embodiments, the excipient is pharmaceutical grade. In some embodiments, the excipient meets US Pharmacopoeia (USP), European Pharmacopoeia (EP), British Pharmacopoeia, and/or International Pharmacopoeia standards.

Pharmaceutically acceptable excipients used in the production of the cell therapy composition include, but are not limited to, inert diluents, dispersants, surfactants and/or emulsifiers, disintegrants, preservatives, buffers, and lubricants. Such excipients can optionally be included in the formulations of the present invention. Excipients such as colorants can be present in the composition at the discretion of the formulator. Exemplary diluents include, but are not limited to, calcium carbonate, sodium carbonate, calcium phosphate, dicalcium phosphate, calcium sulfate, calcium hydrogen phosphate, sodium phosphate, lactose, sucrose, cellulose, microcrystalline cellulose, kaolin, Examples include mannitol, sorbitol, inositol, sodium chloride, dried starch, corn starch, powdered sugar, and the like, and combinations thereof.

Exemplary buffering agents include, but are not limited to, citrate buffer, acetate buffer, phosphate buffer, ammonium chloride, calcium carbonate, calcium chloride, calcium citrate, calcium grubionate, calcium glucoceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, Potassium mixture, dibasic potassium phosphate, primary potassium phosphate, potassium phosphate mixture, sodium acetate, sodium bicarbonate, sodium chloride, sodium citrate, sodium lactate, dibasic sodium phosphate, phosphoric acid Sodium hydrogencarbonate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

In further embodiment, a kit is provided herein the cell transplantation. The kit may comprise buffers for cell washing, a device for cell washing, cells, syringes, needles, cups, containers, alcohol swabs, anesthetics, antibiotics, antioxidants, vitamins, lipids, carbohydrates, hormones, growth factors and more. In certain embodiments, the kit comprises a cannula comprising a funnel shaped hub, tubing, and a pneumatic powered syringe, such as a MICRODOSE™ syringe. In certain embodiments, the components of the kit are sterilized and packaged for convenient use by a surgeon or other healthcare professional or during the manufacturing process. The kit can also include instructions for using the cannula and other agents in the transplant procedure. The kit can provide the components necessary for a single use. The kit can also include packaging materials and information required by government supervisors that regulate pharmaceuticals and/or medical devices.

### VI. Ocular Cells

In some embodiments, retinal progenitor cells (RPEs), photoreceptors and/or photoreceptor precursor cells are delivered by the apparatus provided herein. The cells in the retina that are directly sensitive to light are the photoreceptor cells. Photoreceptors are photosensitive neurons in the outer part of the retina and can be either rods or cones. In the process of phototransduction, the photoreceptor cells convert incident light energy focused by the cornea and lens to electric signals which are ultimately sent via the optic nerve to the brain. Vertebrates have two types of photoreceptor cells including cones and rods. Cones are adapted to detect fine detail, central and color vision and function well in bright light. Rods are responsible for peripheral and dim light vision. Neural signals from the rods and cones undergo processing by other neurons of the retina.

Photoreceptors can express markers such as OTX2, CRX, PRDM1 (BLIMP1), NEUROD1, RCVRN, TUB B3 and L1CAM (CD171). Photoreceptors express several proteins that can serve as markers for detection by the use of methodologies, such as immunocytochemistry, Western blot analysis, flow cytometry, or enzyme-linked immunoassay (ELISA). For example, one characteristic photoreceptor-marker is RCVRN. Photoreceptors may not express (at any detectable level) the embryonic stem cells markers OCT-4, NANOG or REX-1. Specifically, expression of these genes is approximately 100-1000 fold lower in photoreceptors than in ES cells or iPSC cells, when assessed by quantitative RT-PCR.

Photoreceptor markers may be detected at the mRNA level, for example, by reverse transcriptase polymerase chain reaction (RT-PCR), Northern blot analysis, microarray, or RNA-sequencing including single-cell RNA sequencing dot-blot hybridization analysis using sequence-specific primers in standard amplification methods using publicly available sequence data (GENBANK®). Expression of tissue-specific markers as detected at the protein or mRNA level is considered positive if the level is at least or about 2-, 3-, 4-, 5-, 6-, 7-, 8-, or 9-fold, and more particularly more than 10-, 20-, 30, 40-, 50-fold or higher above that of a control cell, such as an undifferentiated pluripotent stem cell or other unrelated cell type.

Dysfunction, injury and loss of photoreceptor cells are factors of many eye diseases and disorders including age-related macular degeneration (AMD), hereditary macular degenerations including Best disease, Stargardt disease and choroideremia, retinitis pigmentosa, and other forms of inherited retinal diseases and acquired retinal dysfunctions, diseases, and injuries. A potential treatment for such diseases is the transplantation of PRP and/or PR into the retina of those in need of such treatment. It is speculated that the replenishment of PRP and/or PR by their transplantation may delay, halt or reverse degradation, improve retinal function and prevent blindness stemming from such conditions. However, obtaining PRP and/or PR directly from human donors and embryos is a challenge.

The retinal pigment epithelium acts as party of the barrier between the bloodstream and the retina and closely interacts with photoreceptors in the maintenance of visual function and choroidal blood supply. The retinal pigment epithelium is composed of a single layer of hexagonally shaped cells that are densely packed with granules of melanin. The main functions of the specialized RPE cells include: transport of nutrients such as glucose, retinol, and fatty acids from the blood to the photoreceptors; transport of water, metabolic end products, and ions from the subretinal space to the blood; absorption of light and protection against photooxidation; reisomerization of all-trans-retinol into 11-cis-retinal; phagocytosis of shed photoreceptor membranes; and secretion of various essential factors for the structural integrity of the retina.

Mature retinal pigment epithelium expresses markers such as cellular retinaldehyde-binding protein (CRALBP), RPE65, best vitelliform macular dystrophy gene (VMD2), and pigment epithelium derived factor (PEDF). Malfunction of the retinal pigment epithelium is associated with a number of vision-altering conditions, such as retinal pigment epithelium detachment, dysplasia, atrophy, retinopathy, retinitis pigmentosa, macular dystrophy, or degeneration, including age-related macular degeneration.

Mature retinal pigment epithelial (RPE) cells can be characterized based upon their pigmentation, epithelial morphology, and apical-basal polarity. Differentiated RPE cells can be visually recognized by their cobblestone morphology and the initial appearance of pigment. In addition, differentiated RPE cell layer have transepithelial resistance/TER, and generates trans-epithelial potential/TEP across the monolayer (TER>100 ohms. cm2; TEP>2 mV), transport fluid, lactic acid, and CO₂ from the apical to basal side, and regulate a polarized secretion of cytokines.

RPE cells express several proteins that can serve as markers for detection of their identity and maturation state by the use of methodologies such as immunocytochemistry, Western blot analysis, flow cytometry, and enzyme-linked immunoassay (ELISA). For example, RPE-specific markers may include: cellular retinaldehyde binding protein (CRALBP), microphthalmia-associated transcription factor (MITF), tyrosinase-related protein 1 (TYRP-1), retinal pigment epithelium-specific 65 kDa protein (RPE65), premelanosome protein (PMEL17), bestrophin 1 (BEST1), and c-mer proto-oncogene tyrosine kinase (MERTK). At the same time, RPE cells do not express (at any detectable level) the embryonic stem cells markers Oct-4, nanog or Rex-1. Specifically, expression of these genes is approximately 100-1000 fold lower in RPE cells than in ES cells or iPSC cells, when assessed by quantitative RT-PCR.

RPE cell markers may be detected at the mRNA level, for example, by reverse transcriptase polymerase chain reaction (RT-PCR), Northern blot analysis, or dot-blot hybridization analysis using sequence-specific primers in standard amplification methods using publicly available sequence data (GENBANK®). Expression of tissue-specific markers as detected at the protein or mRNA level is considered positive if the level is at least or about 2-, 3-, 4-, 5-, 6-, 7-, 8-, or 9-fold, and more particularly more than 10-, 20-, 30, 40-, 50-fold or higher above that of a control cell, such as an undifferentiated pluripotent stem cell or other unrelated cell type.

Dysfunction, injury, and loss of RPE cells are factors of many eye diseases and disorders including age-related macular degeneration (AMD), hereditary macular degenerations including Best disease, Stargardt disease and choroideremia, and other forms of inherited retinal diseases and acquired retinal dysfunctions, diseases, and injuries, including but not limited to RPE tears/rips. A potential treatment for such diseases is the transplantation of RPE cells into the subretinal space of those in need of such treatment. It is speculated that the replenishment of RPE cells by their transplantation may delay, halt or reverse degradation, improve retinal function and prevent blindness stemming from such conditions. However, obtaining RPE cells directly from human donors and embryos is challenging.

In some aspects, RPE cells are produced from iPSCs, such as by the method disclosed in PCT/US2016/050543 and PCT/US2016/050554.

In some embodiments, methods are provided for producing photoreceptors from an essentially single cell suspension of PSCs such as human iPSCs. In some embodiments, the PSCs are cultured to pre-confluence. In certain aspects, the PSCs are dissociated by incubation with a cell dissociation solution or enzyme, such as exemplified by Versene, Trypsin, ACCUTASE™ or TRYPLE™. PSCs can also be dissociated into an essentially single cell suspension by pipetting.

In addition, Blebbistatin (e.g., about 2.5 μM) can be added to the medium to increase PSC survival after dissociation into single cells while the cells are not adhered to a culture vessel. A ROCK inhibitor instead of Blebbistatin may alternatively be used to increase PSC survival after dissociation into single cells.

Once a single cell suspension of PSCs is obtained, the cells are generally seeded in an appropriate culture vessel, such as a tissue culture plate, such as a flask, multi-layer flask, 6-well, 12-well, 24-well, 96-well or 10 cm plate. A culture vessel used for culturing the cell(s) can include, but is particularly not limited to: flask, flask for tissue culture, dish, Petri dish, dish for tissue culture, multi dish, micro plate, micro-well plate, multi plate, multi-well plate, micro slide, chamber slide, tube, tray, CELLSTACK® Chambers, culture bag, and roller bottle, as long as it is capable of culturing the stem cells therein. The cells may be cultured in a volume of at least or about 0.2, 0.5, 1, 2, 5, 10, 20, 30, 40, 50 ml, 100 ml, 150 ml, 200 ml, 250 ml, 300 ml, 350 ml, 400 ml, 450 ml, 500 ml, 550 ml, 600 ml, 800 ml, 1000 ml, 1500 ml, or any range derivable therein, depending on the needs of the culture. In a certain embodiment, the culture vessel may be a bioreactor, which may refer to any device or system ex vivo that supports a biologically active environment such that cells can be propagated. The bioreactor may have a volume of at least or about 2, 4, 5, 6, 8, 10, 15, 20, 25, 50, 75, 100, 150, 200, 500 liters, 1, 2, 4, 6, 8, 10, 15 cubic meters, or any range derivable therein.

In certain aspects, the PSCs, such as iPSCs, are plated at a cell density appropriate for efficient differentiation. Generally, the cells are plated at a cell density of about 1,000 to about 75,000 cells/cm², such as of about 5,000 to about 40,000 cells/cm². In a 6 well plate, the cells may be seeded at a cell density of about 50,000 to about 400,000 cells per well. In exemplary methods, the cells are seeded at a cell density of about 100,000, about 150,000, about 200,000, about 250,000, about 300,000 or about 350,000 cells per well, such as about 50,000 cells per well.

The PSCs, such as iPSCs, are generally cultured on culture plates coated by one or more cellular adhesion proteins to promote cellular adhesion while maintaining cell viability. For example, preferred cellular adhesion proteins include extracellular matrix proteins such as vitronectin, laminin, collagen, and/or fibronectin, which may be used to coat a culturing surface as a means of providing a solid support for pluripotent cell growth. The term “extracellular matrix (ECM)” is recognized in the art. Its components can include, but are not limited to, one or more of the following proteins: fibronectin, laminin, vitronectin, tenascin, entactin, thrombospondin, elastin, gelatin, collagen, fibrillin, merosin, anchorin, chondronectin, link protein, bone sialoprotein, osteocalcin, osteopontin, epinectin, hyaluronectin, undulin, epiligrin, and kalinin. Other ECM components may include synthetic peptides for adhesion (e.g., RGD or IKVAV motifs), synthetic hydrogels (e.g., PEG, PLGA, etc.) or natural hydrogels, such as alginate. In exemplary methods, the PSCs are grown on culture plates coated with vitronectin. In some embodiments, the cellular adhesion proteins are human proteins.

The extracellular matrix proteins may be of natural origin and purified from human or animal tissues or, alternatively, the ECM proteins may be genetically engineered recombinant proteins or synthetic in nature. The ECM proteins may be a whole protein or in the form of peptide fragments, native or engineered. Examples of ECM protein that may be useful in the matrix for cell culture include laminin, collagen I, collagen IV, fibronectin and vitronectin. In some embodiments, the matrix composition is xeno-free. For example, in the xeno-free matrix to culture human cells, matrix components of human origin may be used, wherein any non-human animal components may be excluded.

In some aspects, the total protein concentration in the matrix composition may be about 1 ng/mL to about 1 mg/mL. In some preferred embodiments, the total protein concentration in the matrix composition is about 1 μg/mL to about 300 μg/mL. In more preferred embodiments, the total protein concentration in the matrix composition is about 5 μg/mL to about 200 μg/mL.

Cells, such as photoreceptors or PSCs, can be cultured with the nutrients necessary to support the growth of each specific population of cells. Generally, the cells are cultured in growth media including a carbon source, a nitrogen source and a buffer to maintain pH. The medium can also contain fatty acids or lipids, amino acids (such as non-essential amino acids), vitamin(s), growth factors, cytokines, antioxidant substances, pyruvic acid, buffering agents, pH indicators, and inorganic salts. An exemplary growth medium contains a minimal essential media, such as Dulbecco's Modified Eagle's medium (DMEM) or ESSENTIAL 8™ (E8™) medium, supplemented with various nutrients, such as non-essential amino acids and vitamins, to enhance stem cell growth. Examples of minimal essential media include, but are not limited to, Minimal Essential Medium Eagle (MEM) Alpha medium, Dulbecco's modified Eagle medium (DMEM), RPMI-1640 medium, 199 medium, and F12 medium. Additionally, the minimal essential media may be supplemented with additives such as horse, calf or fetal bovine serum. Alternatively, the medium can be serum free. In other cases, the growth media may contain “knockout serum replacement,” referred to herein as a serum-free formulation optimized to grow and maintain undifferentiated cells, such as stem cell, in culture. KNOCKOUT™ serum replacement is disclosed, for example, in U.S. Patent Application No. 2002/0076747, which is incorporated herein by reference. Preferably, the PSCs are cultured in a fully-defined and feeder-free media.

Accordingly, the single cell PSCs are generally cultured in a fully defined culture medium after plating. In certain aspects, about 18-24 hours after seeding, the medium is aspirated and fresh medium, such as E8™ medium, is added to the culture. In certain aspects, the single cell PSCs are cultured in the fully defined culture medium for about 1, 2 or 3 days after plating. Preferably, the single cells PSCs are cultured in the fully defined culture medium for about 2 days before proceeding with the differentiation process.

In some embodiments, the medium may contain or may not contain any alternatives to serum. The alternatives to serum can include materials which appropriately contain albumin (such as lipid-rich albumin, albumin substitutes such as recombinant albumin, plant starch, dextrans and protein hydrolysates), transferrin (or other iron transporters), fatty acids, insulin, collagen precursors, trace elements, 2-mercaptoethanol, 3′-thioglycerol, or equivalents thereto. The alternatives to serum can be prepared by the method disclosed in International Publication No. WO 98/30679, for example. Alternatively, any commercially available materials can be used for more convenience. The commercially available materials include KNOCKOUT™ Serum Replacement (KSR), Chemically-defined Lipid concentrated (Gibco), and GLUTAMAX™ (Gibco).

Other culturing conditions can be appropriately defined. For example, the culturing temperature can be about 30 to 40° C., for example, at least or about 31, 32, 33, 34, 35, 36, 37, 38, 39° C. but particularly not limited to them. In one embodiment, the cells are cultured at 37° C. The CO₂ concentration can be about 1 to 10%, for example, about 2 to 5%, or any range derivable therein. The oxygen tension can be at least, up to, or about 1, 2, 3, 4, 5, 6, 7, 8, 9, 10, 20%, or any range derivable therein.

### VII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Delivery of Photoreceptor Precursor Cell Aggregates

Studies were conducted to develop a dose delivery procedure for both large animal and clinical settings. Allogeneic human induced pluripotent stem cell (iPSC)-derived photoreceptor precursor cell (iPRP) aggregates were developed as a cell-based PR replacement therapy. After dose preparation (REP-00798), iPRP aggregates were transplanted using Alcon's Constellation Vision System [1], an ophthalmic microsurgical system. The MICRODOSE™ Injection Kit 1 mL syringe (MedOne Surgical, Inc., # 3275) [2] was attached to the CONSTELLATION® Vision System (Alcon) via tubing from the Viscous Fluid Control (VFC) pack (#8065750957). Studies were conducted to identify the most appropriate dose loading and delivery procedure, including generation and testing of a novel cannula hub geometry for delivery of the cell product, such as an aggregate cell product.

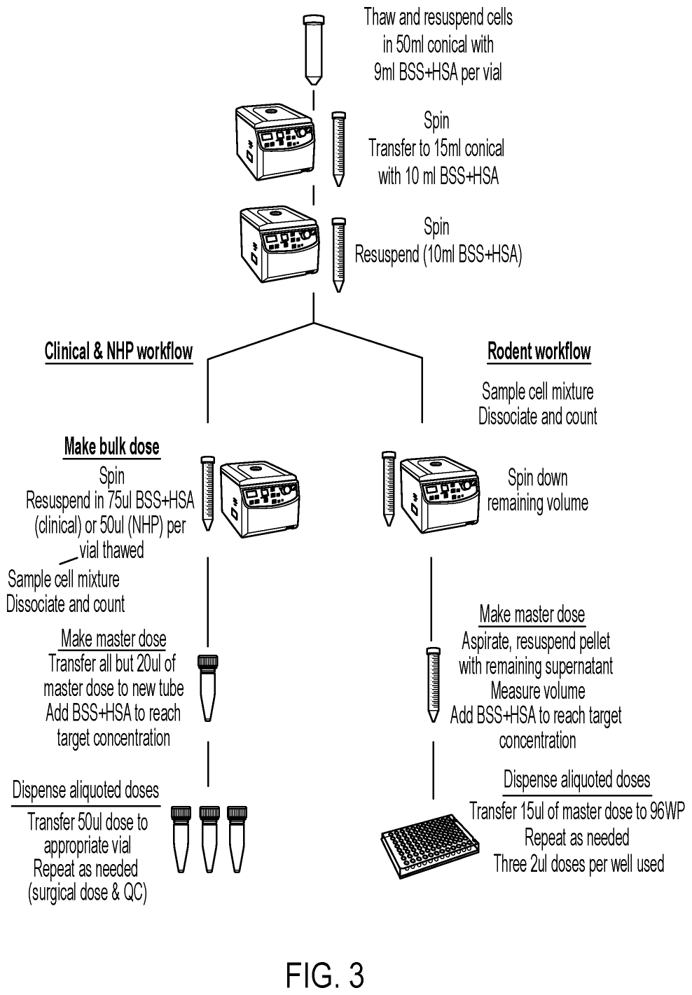

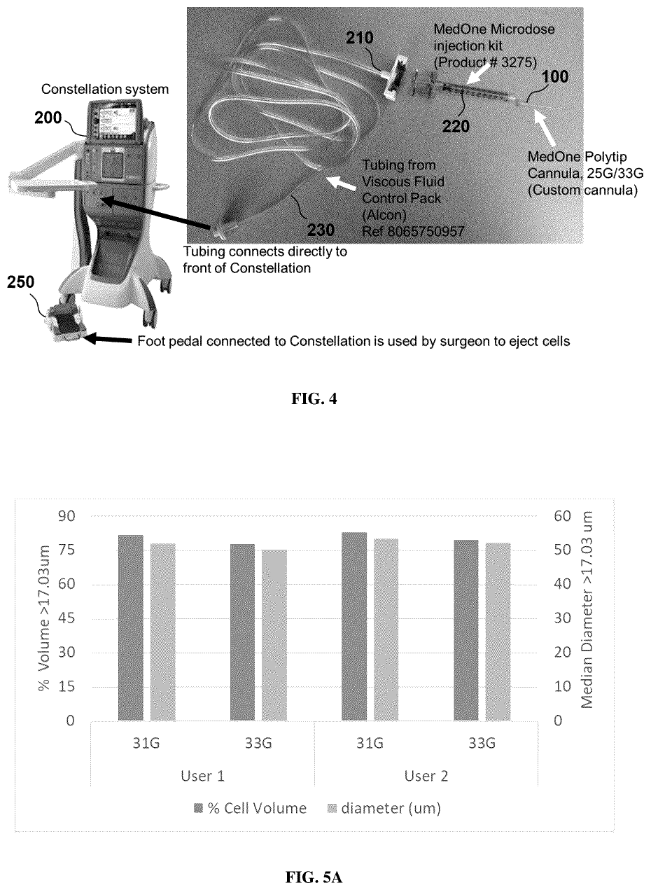

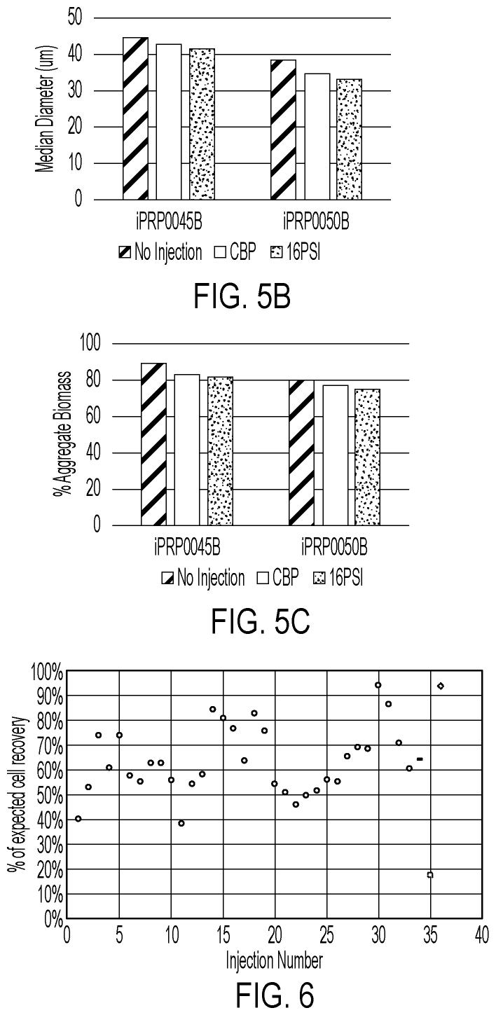

Dose formulation. To prepare iPRP aggregates for transplant, cells were thawed and resuspended in BSS with 0.2% HSA buffer, unless otherwise noted. Multiple spin and wash steps followed, along with removal of a sample for dissociation with 10X TrypLE and cell counting. Using the viable cell concentration, dose formulations were made (FIG. 3).

Dose analysis. For the bench testing, doses were either loaded into a 1 mL syringe (BD # 309628 or MedOne Surgical, Inc., #3275) through an 18G needle and then ejected through a 31G (MedOne #3218) or 33G (MedOne #3262) cannula (single pass method) or loaded into a syringe through a 31G (MedOne #3218) or 33G (MedOne #3262) cannula and then ejected through the same cannula (dual pass method). Pressure to pull up and eject the dose was either generated manually with a plunger (BD #309628) or through attachment of the 1 mL syringe (MedOne Surgical, Inc., #3275) to the CONSTELLATION® Vision System via the Viscous Fluid Control (VFC) pack (#8065750957).

Once ejected, in order to analyze the number of cells recovered, the aggregates were enzymatically dissociated in 10X TrypLE for 30 minutes, quenched in DMEM/F12+B27+Benzonase (iPRP Quench Medium), triturated, and counted. Alternatively, to access aggregate integrity, aggregates were transferred to the Multisizer 4 and run through a 280 μm aperture.

iPRP aggregate device structure overview. To deliver iPRP aggregates to the subretinal space of large animals or to humans in the clinic, the CONSTELLATION® Vision System (Alcon, FIG. 4) [1] was used to load and inject volumes at a controlled pressure. The MICRODOSE™ Injection Kit 1 mL syringe (MedOne Surgical, Inc., #3275) [2] was attached to the CONSTELLATION® Vision System via tubing from the Viscous Fluid Control (VFC) pack (#8065750957). A injection attachment 100 (MedOne Polytip Cannula, 25G/33G was connected to the syringe through which a 50 μL volume of cells was delivered into the subretinal space via a pre-defined uniform injection pressure (psi), controlled by a foot pedal.

Comparison of a 31G vs 33G cannula. The integrity of the iPRP aggregates can be negatively impacted by how it is handled. Therefore, in order to decrease the shear forces during loading and injection of the dose, a larger diameter cannula may be used. However, because the cannula must fit through a retinotomy to deliver the dose behind the retina, a smaller cannula may be needed to minimize the amount of damage to the retina. Additionally, a larger retinotomy (to allow for a smaller gauge cannula) creates a wider hole in the retina which increases the incidence of injected iPRP aggregate reflux out of the bleb, subsequently delivering less product. Historically, a 31 Ga cannula had been used, but with the potential positive impact on the surgical procedure, the present studies investigated whether a smaller diameter cannula (33G) would negatively impact the aggregate product.

To test the impact of cannula gauge on aggregate integrity, iPRP aggregates were thawed and prepared. 200 μL of final aggregate product formulation was manually loaded into the syringe through an 18G needle. The needle was then replaced by either a MedOne 31G (#3218) or 33G POLYTIP® Cannula (#3262). The trapped air was removed from the syringe and cannula and the unused portion of the dose was ejected until the plunger reached the 50 μL gradation (the volume of the clinical dose). The 50 μL dose was then ejected and aggregate size analyzed on the Multisizer (280 μm aperture). A gate was drawn from ˜17 μm to 168 μm to capture the aggregates, and the percent volume of the product that fell within that gate was measured. Additionally, the median aggregate diameter was calculated within that gate. Based on these results, using a 33G cannula should be comparable to the 31G POLYTIP® Cannula in terms of aggregate integrity, as no appreciable difference was seen in the volume of aggregates or median diameter. Because of the negligible impact of cannula diameter on aggregate integrity and the benefits to the surgery when using the smaller cannula, further studies were conducted with the 33G POLYTIP® Cannula (#3262).

An investigation into variable surgical dose deliveries. Because the iPRPs are composed of aggregates, it settles out of the formulation buffer quickly (REP-00798). This not only impacts dose formulation but could also create problems during dose delivery. Testing was therefore performed to determine if product settling in the syringe might lead to inaccurate dosing.

Historically, in vitro experiments during this time used a manual 1 mL syringe which was attached to an 18G needle during loading (200 μL), which was removed, and cells were ejected through a 33G cannula (50 μL). The 50 ul injection of an iPRP aggregate dose was then dissociated in 10X TrypLE and counted. It should be noted that the experiments laid out here were originally intended to investigate different formulation vehicles (BSS Plus, BSS Part I only, BSS Plus with Benzonase, BSS Plus with 0.2% HSA, BSS Plus with 0.2% HSA and Benzonase, Standard BSS, and RMN), however as there was no obvious impact of vehicle, the data was combined for this study. Results are displayed as a percent of expected cell recovery (e.g., 30-100%, 30-50%, 40-60%, 40-90% 50-70%, 60-80%, 70-90%, or 80-100%), which was calculated by dividing the actual viable cells recovered by the targeted dose (FIG. 6, blue circles). After eight experiments across four lots (iPRP0045 20200727-CD133_60, iPRP0047A, iPRP0047B, and iPRP0049A) that totaled 33 individual injections, doses fell within roughly 40-90% of the expected range.

Around this time, a video of a mock surgery with the present cell product, conducted by a surgeon and surgeon assistants, was provided. From this experiment, very few cells were recovered post-injection. Upon reviewing the video, it was noted that the surgery process was slower. Additionally, and importantly, not only was the time increased, but the tip of the cannula was oriented upward for the majority of the time, which likely allowed aggregates to settle toward the syringe plunger preventing them from being injected. To test this hypothesis, three doses of iPRP0047A were generated, one for each tested condition. The control was conducted in the same manner as the previous 8 experiments, where the dose was pulled up and injected quickly (FCDI standard; FIG. 6, dash). Specifically, for the FCDI standard, the cell aggregate composition is resuspended in BSS with 0.2% HSA. The constellation vision system is attached to tubing, a viscous fluid control pack and microdose injection kit syringe. The syringe and cannula is primed with buffer and with the cannula facing up, the foot pedal is depressed to purge excess air and BSA+HSA. The syringe is inserted into the clamp on the ring stand vertically with the cannula tip pointing down. The Constellation system is set to extract the 50 μL dose into the syringe and cannula and allowed to settle for about 5 minutes. The Constellation system is then set to Inject at 10 PSI once the foot peddle is depressed. For the second dose, the scientist mirrored any changes in syringe orientation that the surgeon on the video made from the duration of dose uptake through injection (Surgeon's Orientation; FIG. 6, square). Specifically, the surgeon's orientation comprised holding the dose perpendicular to the ground during settling and for injection with an about 45 degrees angle A expected, as shown in FIG. 17B. To determine if time in the syringe decreased dose recovery, for the third condition, the syringe was loaded, then held for the same length of time as the surgeon's manipulated the dose (approximately 5 minutes), but it was held parallel to the ground and rolled back and forth between the scientist's fingers with the goal of maintaining aggregates in suspension (FIG. 6, diamond).

Once collected, all iPRP aggregate injected doses were dissociated in 10X TrypLE and counted to calculate the percent of expected cell recovery. The surgeon's orientation dose recovery starkly stood out with a recovery of less than 20% (Injection #35; FIG. 6, pink square), while the FCDI standard dose (Injection #34, FIG. 6, green dash) and 5 minute rolled dose (Injection #36, FIG. 6, aqua diamond) had typical recoveries. These findings emphasized the need to either 1) heavily train surgeons and surgeon assistants to maintain the aggregates in suspension or 2) change the delivery approach.

Flicking a loaded syringe is sufficient to maintain aggregate suspension. With the knowledge that iPRP aggregates could fall out of the formulation buffer once loaded into the syringe and therefore result in an inaccurate dose to be injected, it was attempted to identify methods to maintain proper aggregate distribution in the syringe. Three different experiments were conducted using lot iPRP0046C. Doses were prepared such that 2 million cells would be expected to be delivered from the injection. Using the Constellation, 200 μL was loaded into the syringe through an 18G needle. After the 18G needle was replaced with the 33G cannula (MedOne #3262) the air was purged and the extra volume was ejected such that the plunger reached the 50 μL gradation. Typically, at this step in the protocol, the dose is in the device for no longer than 2 minutes prior to injection. To simulate a potential “worst-case scenario”, the syringe was set on its side for 5 minutes, a time at which aggregates were visibly seen to have settled to the side of the syringe. After this wait time, the dose was either immediately ejected (no mixing) or first mixed by either vortexing or flicking the syringe. Over these three experiments, a total of 14 injections were conducted. As expected, mixing prior to dose ejection from the syringe resulted in significant recovery (FIG. 7). Based upon these results, flicking was determined to be sufficient at redistributing the dose.

Taking advantage of aggregate settling by using an injection attachment 100. Flicking syringes loaded with iPRP aggregates redistributed settled aggregates to provide a mixed formulation and reliable injection (FIG. 7). However, concern about variability in surgical injections remained despite these changes, so different methods for dose delivery were tested including purposefully allowing aggregates to settle within the hub of the cannula.

It was hypothesized that if a 50 μL dose was loaded into the syringe and all the aggregates were allowed to settle vertically into the cannula hub, as the dose was injected all of the aggregates should be ejected first, with fewer becoming potentially trapped along the side of the syringe or back against the plunger due to settling and improper resuspension techniques. Therefore, it was next tested whether recovery of aggregates was improved using this new settling method vs the previous suspension method. For this experiment, for the “resuspended” dose, iPRP aggregates were prepared. As in previous experiments, a 200 μL dose was manually pulled up into the syringe through an 18G needle. That 18G needle was then replaced with the 33G cannula (MedOne #3262) and the air and extra volume were removed, leaving a 50 ul dose in the device. This 50 ul dose was then injected into a tube for analysis. However, for the investigation of vertical settling, the 33G cannula was attached to the syringe and both were primed with vehicle (BSS+HSA) to ensure that no air was trapped in the cannula or syringe. Once the air was purged, the plunger was pushed back to 0 μL. The 50 μL dose was then pulled through the cannula with the device held in a vertical orientation (cannula facing down) and a 5-minute wait time commenced. After the incubation time, this 50 μL dose was also ejected into a tube for downstream analysis. The cells were then dissociated and counted to determine the delivered dose. Unfortunately, while the suspension method provided an expected recovery of around 2 million cells, the syringe in which cells were allowed to settle injected less than 25% of the expected dose (FIG. 8A).

A reason for this lack of cell recovery became obvious upon close observation of the cannula hub which, as designed, presents a space in which cells were trapped (FIG. 8B, arrow). A ring of white aggregates was seen in the cannula hub 301 even after the syringe plunger had been completely depressed (FIG. 8C, arrow).

Thus, a injection attachment 100 was developed in which the hub would be shaped like a funnel (FIG. 8D) such that there is limited-to-no space for aggregates to become trapped compared to the off-the-shelf cannula (MedOne#3262, FIG. 8B). Experiments were then conducted with this new cannula design in hopes that the settling method could improve the injected dose consistency versus the suspension method.

Returning to the same experimental design as conducted for FIG. 8A, the off-the-shelf 33G POLYTIP® Cannula (#3262) with the suspension method was compared to the injection attachment 100, with aggregate settling. In the first experiment iPRP0046C, doses of iPRP aggregates was prepared and 7 injections were conducted with the injection attachment 100 following the settling method while 5 injections were made using the off-the-shelf cannula following the suspension method (FIG. 8E). The following experiment was conducted in the exact same manner, with the same iPRP aggregates, but with 4 injection attachment 100 injections and 5 off-the-shelf cannula injections (FIG. 8F). Using the injection attachment 100 with the settling method resulted in CV of 9.2 and 8.4%, respectively while the off-the-shelf cannula showed CVs of 29.2 and 23.4%, respectively. This decreased variability in the injection attachment 100 and settling method eliminated the need to maintain aggregrates in suspension, subsequently alleviating concerns about proper surgical training in order to correctly deliver the iPRP aggregates.

It was noted that in the “resuspension” method, the aggregates passed into the cannula through an 18G needle and out the 33G cannula, while the “settling method” passed the aggregates both through and out the 33G cannula. Since aggregate integrity can be impacted by shear stress, the integrity of aggregates that had been passed through the cannula once or twice was interrogated.

The first experiment was conducted to test the impact two passes through the cannula might have on aggregate integrity. iPRP aggregates were thawed and doses were prepared. Doses were loaded and injected as described above for the suspension method, except that to test a double pass through the cannula, the 200 μL dose was loaded into the syringe through the 33G cannula instead of the 18G needle. After ejection, the 50 μL doses were analyzed on the Multisizer using a 280 μm aperture. A gate was drawn from ˜17 μm to 168 μm and the percent volume of the product that fell within that gate was measured, as well as the median aggregate diameter within that gate. The median aggregate diameter was found to be about 40-60 um, such as 40-45, 45-55, 50-55, or 50-60 um. While a slight decrease in both measurements occurred when aggregates were passed through the 33G cannula twice, the impact was small enough to continue with the settling method (FIG. 9).

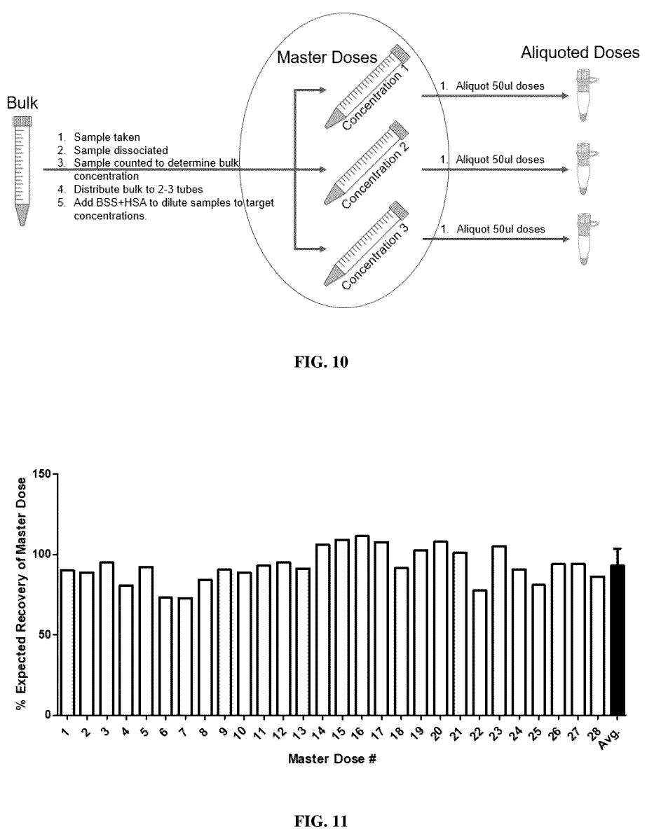

Calculating Dose Variability. For many of the dosing studies, multiple concentrations were tested. For this workflow, the product was thawed, washed, and then resuspended in a set volume of BSS+HSA, which would ensure the Bulk was purposely more concentrated than the final target concentration. A sample was then removed from this Bulk dose and dissociated to obtain cell counts. The Bulk volume was measured, then distributed to multiple tubes, which were diluted in additional BSS+HSA to target the appropriate concentrations. These volumes, called Master Doses, were then dispensed into aliquoted doses used for injection (FIG. 10).

In order to determine the variability between Master Doses, 28 Master Doses from 10 experiments were compared using seven different iPRP aggregate product lots (iPRP0046C, iPRP0047B, iPRP0049C, iPRP0049D, iPRP0054F, iPRP0055A, iPRP0057). To standardize the reporting, the % of expected cell concentration (actual counted concentration divided by target concentration) was plotted. The results showed that these Master Doses, on average, contained 93% of the expected cell concentration with an 11.4% coefficient of variation (FIG. 11). 80% of doses were within±16% of the targeted cell concentration, 90% were within±23% of the target dose with 100% falling within±28% of our dose with a minimum expected cell concentration of 73% and a maximum expected cell concentration of 111% (FIG. 11).

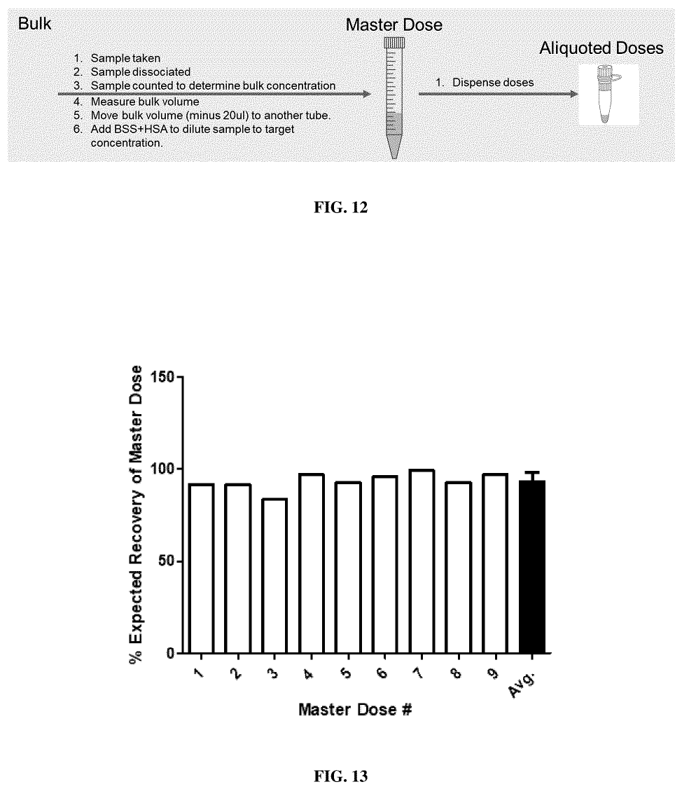

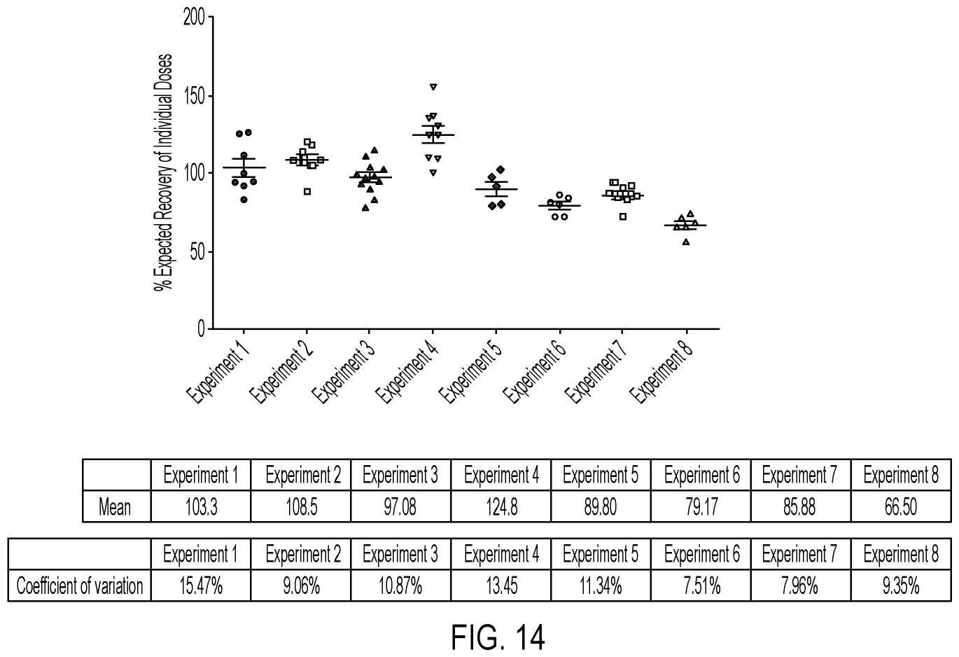

One source of error that might have contributed to variation in these studies was that a single Bulk formulation was used to prepare multiple concentrations of Master Doses. This variability, however, was present in either the clinical or large animal GLP studies, as the protocol calls for only one Master Dose concentration. For these studies the Bulk was prepared, and a sample removed for dissociation and cell counts to calculate the Bulk concentration. However, instead of dividing this bulk across multiple tubes, the entire volume of the Bulk, except 20 μL, was moved to a new tube and diluted to the target concentration (Master Dose). This Master Dose was then dispensed into aliquoted doses for injection (FIG. 12).

Experiments in which one Master Dose was generated from one bulk concentration resulted in lower variability than when multiple Master Doses were generated from the Bulk. Preparation of 9 master doses from 5 different iPRP aggregate lots showed the average Master Dose to have 93.5% of the % of expected cell concentration with a 5% coefficient of variation. 88% of doses were within±10% of our targeted cell concentration, with a minimum expected cell concentration of 84% and a maximum expected cell concentration of 99% (FIG. 13).

Because aggregates settle quickly, variability could be introduced when dispensing the Master Doses into aliquoted doses. In nine experiments that used iPRP aggregates between 5 to 12 aliquots were dispensed containing either 60-70 μL or 210 μL each (this volume overage allowed for each aliquot to contain either 50 μL or 200 μL post-sampling for counts so that they could be used for downstream injection experiments). From each aliquot, 5 μL samples were removed, dissociated, and counted to determine the concentration of the aliquoted dose.

As presented above, % of expected cell concentration was used for this data since different dose concentrations were targeted for these experiments. Importantly, while variation was seen between experiments, dose-to-dose variability within an experiment showed a coefficient of variation range from approximately 7.5% to 15.5%.

Dose compensation factors. The new device loading (the 50 μl dose through the cannula) procedure with settling in an injection attachment 100 provided increased precision, but further studies were performed to assess dosing accuracy. It was observed during multiple studies that fewer injected cells were recovered than anticipated based on the calculated dose concentration. Therefore, a dose compensation factor was considered.

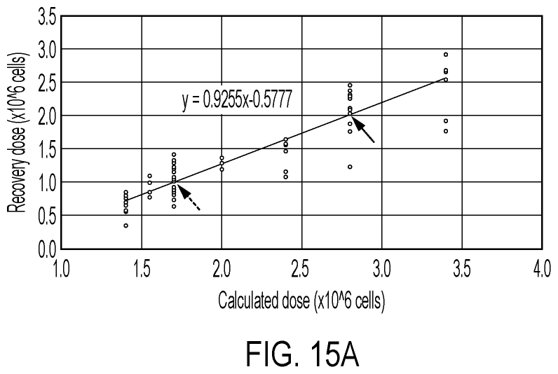

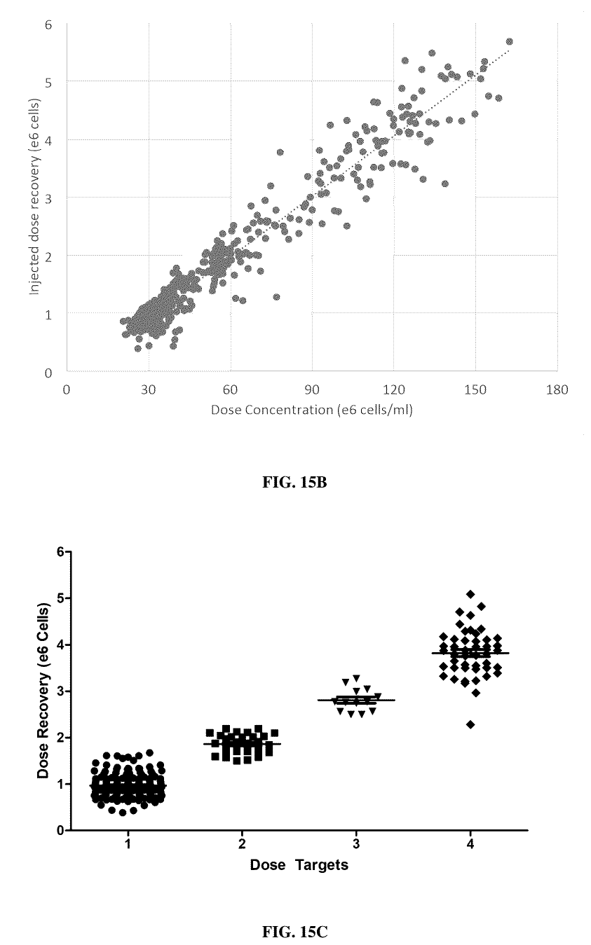

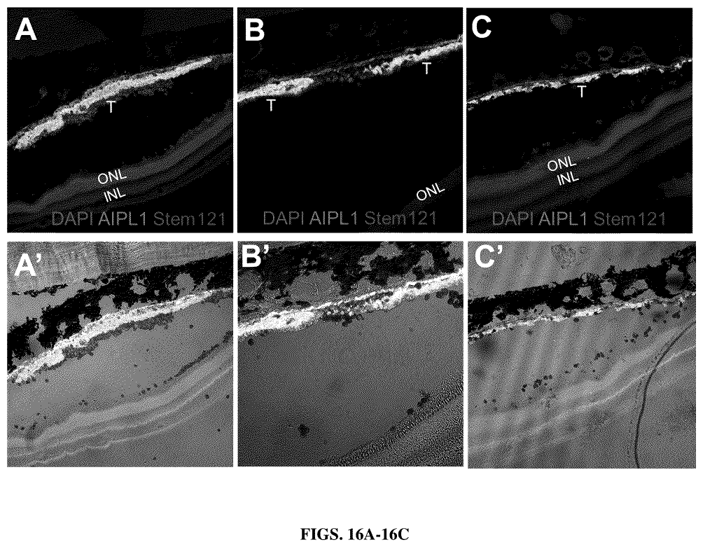

Doses ranging from 1.4-3.4 million cells were prepared and injected using the Constellation across 11 experiments using six different lots of iPRP aggregates (iPRP0047B, iPRP0049C, iPRP0049D, iPRP0054F, iPRP0055A, and iPRP0057) totalling 82 injections. Extrapolating a linear fit line across all data points generated a best fit equation of y=0.9255x−0.5777 (FIG. 15A). Using this equation, it was calculated that doses of 1.7 and 2.8 millioncells loaded were needed to achieve the target doses of one million (clinical) and two million (non-human primate) cells injected, respectively. For the three and four million (clinical) cell doses, a linear fit line was extrapolated across all data points generated for 391 individual injections across 2 iPSC lines, 8 iPRP lots (16 sublots) for all four doses, a best fit equation of 1y=0.0348x−0.1239 was determined (FIG. 15B). Using this equation, it was calculated that doses of 4.5 and 5.95 million cells loaded were needed to achieve the target doses of three and four million cells injected, respectively. The results of testing the 1M, 2M, 3M and 4M dose targets can be seen in FIG. 15C.

Variability of injected clinical doses using the 1.7 million cell compensation was tested in 6 experiments using 3 lots, (iPRP0047B, iPRP0055A, and iPRP0057), with a total of 25 injections. The average dose was 1.04 million cells, with a coefficient of variation (CV) of 19%. 80% of doses were within±27% of the targeted 1 million dose, 90% were within±33% of our dose with 100% falling within±42% of the dose with a minimum dose of 0.64 million cells being injected and a maximim of 1.41 million cells being injected (FIG. 15B). For the non-human primate target of 2 million cells, calculated doses of 2.8 million cells were generated. In 15 injections across 3 experiments and four lots (iPRP0047B, iPRP0054F, iPRP0055A, and iPRP0057), the average dose was 2.1 million cells with a CV of 16%. 80% of doses were within±19% of our targeted 1 million dose, 90% were within±23% of our dose with 100% falling within±38% of our dose with a minimum dose of 1.23 million cells being injected and a maximim of 2.45 million cells being injected (15C).

Observations about and improvements to the large animal (FIG. 16) and clinical iPRP aggregate dose delivery have been described above. The data suggests that with the implemented changes, a more reliable dose may be delivered upon each injection.

The initial protocol required loading the cells through an 18G needle, which was then replaced with a 31G cannula prior to injection. While this protocol worked, improvements were possible. The 31G cannula was sufficient to deliver cells subretinally, however, it required a large retinotomy, with the possibility of more retinal damage and the possibility of iPRP aggregate reflux. Testing of the 33G cannula, which had a smaller diameter, showed that the increased shear stress on the aggregates had minimal effect on the aggregate size distribution. Thus, further studies were conducted with the 33G cannula.

Subsequent testing informed whether aggregates could pass through the cannula twice without consequential damage. As no impact on aggregate integrity was observed, the dose loading was converted to the 33G cannula, instead of the 18G needle. Decreasing the handling steps not only made the process easier, but also decreased the chance of contamination and needle sticks.

This dual pass method also allowed for an overhaul of the syringe handling prior to dose delivery. The single-pass method required that a 200 μL dose be loaded into the syringe to help aid in the removal of air in the syringe and cannula. Once the air was removed, volume was dispensed until a 50 ul dose remained. To prevent aggregate settling, both the loading process and time between loading and delivering needed to be performed quickly with this method, and even then, variation in the cells delivered during surgeries was seen. For this reason, it was investigated purposefully allowing aggregates to settle, in hopes of injecting a most consistent dose.

While allowing aggregates to settle toward the cannula tip failed to work initially due to the geometry of the cannula hub, an injection attachment 100 design, which used a new hub—that acted more like a funnel—provided a much more consistent dose. With this newly designed cannula, a 50 ul dose could be loaded through the cannula, allowed to settle toward its tip for 5 minutes, and then injected, eliminating the need to keep aggregates in suspension prior to injection. In addition to improved dose consistency, it should be noted that this method uses a quarter of the dose that the original method used (50 μL vs 200 μL).

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 18. The other clauses can be presented in a similar manner.

Clause 1. A method for cell transplantation comprising injecting cells to a tissue of a subject using an injection attachment comprising a cannula with a funnel shaped hub.

Clause 2. The method of clause 1, wherein the cannula hub is less than 30 mm in length.

Clause 3. The method of clause 1 or 2, wherein the cannula comprises a cannula tip that is 30 gauge or smaller.

Clause 4. The method of clause 3, wherein the cannula tip is blunt.

Clause 5. The method of clause 3, wherein the cannula tip is sharp.

Clause 6. The method of clause 5, wherein the cannula tip is further defined as a cannula tip.

Clause 7. The method of clause 3, wherein the cannula tip is a 34, 33, 32, 31, or 30 gauge cannula tip.

Clause 8. The method of clause 3, wherein the cannula tip is a 33 gauge cannula tip.

Clause 9. The method of any of clauses 3-8, wherein the cells are loaded into the cannula with a cannula tip that is 30 gauge or smaller.

Clause 10. The method of any of clauses 3-8, wherein the cells are loaded into the cannula with a 33 gauge cannula tip.

Clause 11. The method of any of clauses 3-8, wherein the cells are loaded into the cannula and injected to the tissue of said subject with the same cannula tip.

Clause 12. The method of any of clauses 1-11, wherein the cannula tip is not changed between loading into the cannula and injected to the tissue of said subject.

Clause 13. The method of any of clauses 1-12, wherein the wherein the cannula tip is made of a flexible polyimide material or metal.

Clause 14. The method of any of clauses 1-13, wherein the cannula tip is flexible.

Clause 15. The method of any of clauses 1-13, wherein the cannula tip is rigid.

Clause 16. The method of any of clauses 1-7, wherein the hub comprises a one-way check valve.

Clause 17. The method of any of clauses 1-16, wherein the hub comprises a coupling mechanism.

Clause 18. The method of clause 17, wherein the coupling mechanism is a luer lock.

Clause 19. The method of clause 18, wherein the luer lock is connected to a dosing mechanism.

Clause 20. The method of clause 19, wherein the doing mechanism is a syringe.

Clause 21. The method of clause 20, wherein the syringe is a microinjection syringe.

Clause 22. The method of clause 20, wherein the syringe is further connected to tubing.

Clause 23. The method of clause 22, wherein the tubing is connected to a pressure control system.

Clause 24. The method of any of clauses 1-23, wherein the cells are delivered from the injection attachment at a controlled pressure.

Clause 25. The method of any of clause 1-24, wherein the cells are not redistributed prior to loading to the injection attachment.

Clause 26. The method of any of clauses 1-25, wherein the cells are redistributed prior to loading to the injection attachment.

Clause 27. The method of clause 26, wherein the cells are redistributed by vortexing or manual agitation.

Clause 28. The method of any of clauses 1-27, wherein the cells are injected to an eye of said subject.

Clause 29. The method of clause 28, wherein the cells are injected subretinally.

Clause 30. The method of any of clauses 1-29, wherein the cells are further defined as cell aggregates.

Clause 31. The method of any of clauses 1-29, wherein the cells are further defined as single cells.

Clause 32. The method of any of clauses 1-31, wherein the cells are in a formulation buffer.

Clause 33. The method of clause 32, wherein the formulation buffer is a balanced salt solution.

Clause 34. The method of clause 33, wherein the balanced salt solution further comprises benzonase and/or human serum albumin.

Clause 35. The method of clause 30, wherein the cell aggregates are not in suspension when injected to the tissue of said subject.

Clause 36. The method of clause 35, wherein the cells are present in the cannula for at least 5 minutes between loading and injecting to allow aggregate settling in the cannula hub.

Clause 37. The method of clause 35, wherein the cells are present in the cannula for at least 5 minutes between loading and injecting to allow cell settling in the cannula hub.

Clause 38. The method of any of clauses 35-37, wherein the cannula is pointed down to allow cell setting in the cannula hub.

Clause 39. The method of any of clauses 30-36, wherein the cell aggregates are photoreceptor precursor cell aggregates.

Clause 40. The method of any of clauses 1-36, wherein the cells are retinal progenitor cells and/or photoreceptor precursor cells.

Clause 41. The method of any of clauses 30-36, wherein the cell aggregates are retinal progenitor cells and/or photoreceptor precursor cells.

Clause 42. The method of any of clauses 1-39, wherein the cells are injected in a volume of less than 200 μL

Clause 43. The method of any of clauses 1-42, wherein the cells are injected in a volume less than 100 μL

Clause 44. The method of any of clauses 1-43, wherein the cells are injected in a volume of about 50 μL.

Clause 45. The method of any of clauses 1-44, wherein at least 1 million cell are injected.

Clause 46. The method of any of clauses 1-45, wherein at least 2 million cells are injected.

Clause 47. The method of any of clauses 1-46, wherein at least 25% of the cells that are loaded into the injection attachment are injected to the tissue of said subject.

Clause 48. The method of any of clauses 1-47, wherein at least 30% of the cells that are loaded into the injection attachment are injected to the tissue of said subject.

Clause 49. A method for treating an ocular condition in a subject comprising cell transplantation in the eye of the subject using an injection attachment comprising a cannula with a funnel shaped hub.

Clause 50. The method of clause 49, wherein the cannula hub is less than 30 mm in length.

Clause 51. The method of clause 49 or 50, wherein the cannula comprises a cannula tip that is 30 gauge or smaller.

Clause 52. The method of clause 51, wherein the cannula tip is blunt.

Clause 53. The method of clause 51, wherein the canula tip is sharp.

Clause 54. The method of clause 53, wherein the cannula tip is further defined as a needle tip.

Clause 55. The method of clause 51, wherein the cannula tip is a 34, 33, 32, 31, or 30 gauge cannula tip.

Clause 56. The method of clause 51, wherein the cannula tip is a 33 gauge cannula tip.

Clause 57. The method of any of clauses 51-56, wherein the cells are loaded into the cannula with a cannula tip that is 30 gauge or smaller.

Clause 58. The method of any of clauses 51-56, wherein the cells are loaded into the cannula with a 33 gauge cannula tip.

Clause 59. The method of any of clauses 51-56, wherein the cells are loaded into the cannula and injected to the eye of said subject with the same cannula tip.

Clause 60. The method of any of clauses 49-59, wherein the cannula tip is not changed between loading into the cannula and injected to the eye of said subject.

Clause 61. The method of any of clauses 49-60, wherein the wherein the cannula tip is made of a flexible polyimide material or metal.

Clause 62. The method of any of clauses 49-61, wherein the cannula tip is flexible.

Clause 63. The method of any of clauses 49-61, wherein the cannula tip is rigid.

Clause 64. The method of any of clauses 49-55, wherein the hub comprises a one-way check valve.

Clause 65. The method of any of clauses 49-64, wherein the hub comprises a coupling mechanism.

Clause 66. The method of clause 65, wherein the coupling mechanism is a luer lock.

Clause 67. The method of clause 66, wherein the luer lock is connected to a dosing mechanism.

Clause 68. The method of clause 67, wherein the doing mechanism is a syringe.

Clause 69. The method of clause 68, wherein the syringe is a microinjection syringe.

Clause 70. The method of clause 68, wherein the syringe is further connected to tubing.

Clause 71. The method of clause 70, wherein the tubing is connected to a pressure control system.

Clause 72. The method of any of clauses 49-71, wherein the cells are delivered from the injection attachment at a controlled pressure.

Clause 73. The method of any of clause 49-72, wherein the cells are not redistributed prior to loading to the injection attachment.

Clause 74. The method of any of clauses 49-73, wherein the cells are redistributed prior to loading to the injection attachment.

Clause 75. The method of clause 74, wherein the cells are redistributed by vortexing or manual agitation.

Clause 76. The method of any of clauses 49-76, wherein the cells are injected subretinally.

Clause 77. The method of any of clauses 49-76, wherein the cells are further defined as cell aggregates.

Clause 78. The method of any of clauses 49-76, wherein the cells are further defined as single cells.

Clause 79. The method of any of clauses 49-78, wherein the cells are in a formulation buffer.

Clause 80. The method of clause 79, wherein the formulation buffer is a balanced salt solution.

Clause 81. The method of clause 80, wherein the balanced salt solution further comprises benzonase and/or human serum albumin.

Clause 82. The method of clause 77, wherein the cell aggregates are not in suspension when injected to the tissue of said subject.

Clause 83. The method of clause 82, wherein the cells are present in the cannula for at least 5 minutes between loading and injecting to allow aggregate settling in the cannula hub.

Clause 84. The method of clause 82, wherein the cells are present in the cannula for at least 5 minutes between loading and injecting to allow cell settling in the cannula hub.

Clause 85. The method of any of clauses 82-84, wherein the cannula is pointed down to allow cell setting in the cannula hub.

Clause 86. The method of any of clauses 77-83, wherein the cell aggregates are photoreceptor precursor cell aggregates.

Clause 87. The method of any of clauses 49-83, wherein the cells are retinal progenitor cells and/or photoreceptor precursor cells.

Clause 88. The method of any of clauses 77-83, wherein the cell aggregates are retinal progenitor cells and/or photoreceptor precursor cells.

Clause 89. The method of any of clauses 49-86, wherein the cells are injected in a volume of less than 200 μL.

Clause 90. The method of any of clauses 49-89, wherein the cells are injected in a volume less than 100 μL.

Clause 91. The method of any of clauses 49-90, wherein the cells are injected in a volume of about 50 μL.

Clause 92. The method of any of clauses 49-91, wherein at least 1 million cell are injected.

Clause 93. The method of any of clauses 49-92, wherein at least 2 million cells are injected.

Clause 94. The method of any of clauses 49-93, wherein at least 25% of the cells that are loaded into the injection attachment are injected to the eye of said subject.

Clause 95. The method of any of clauses 49-94, wherein the ocular condition is inherited retinal disease, age-related macular degeneration (AMD), inherited macular degeneration, Stargardt's macular dystrophy, Best disease, choroideremia, diabetic retinopathy, retinal vascular disease, damage caused by retinopathy pf prematurity (ROP), or viral infection of the eye.

Clause 96. A cannula apparatus for cell translation comprising:
a tubular hub, wherein the tubular hub comprises:
an outer wall;
an inner wall;
a first end; and
a second end, wherein the first end has a funnel shape sloped towards the second end.

Clause 97. The apparatus of clause 96, wherein the inner diameter of the tubular hub is decreasing as measured from the first end towards the second end.

Clause 98. The apparatus of clause 96, wherein the hub has essentially no catch points.

Clause 99. The apparatus of clause 96, wherein the hub has a smooth surface.

Clause 100. The apparatus of clause 96 or 97, wherein the tubular hub is less than 30 mm in length.

Clause 101. The apparatus of any of clauses 96-100, wherein the first end is attached to a cannula tip.

Clause 102. The apparatus of clause 101, wherein the cannula tip is blunt.

Clause 103. The apparatus of clause 102, wherein the cannula tip is sharp.

Clause 104. The apparatus of clause 103, wherein the cannula tip is further defined as a needle tip.

Clause 105. The apparatus of clause 101, wherein the cannula tip is 30 gauge or smaller.

Clause 106. The apparatus of clause 105, wherein the cannula tip is a 34, 33, 32, 31, or 30 gauge cannula tip.

Clause 107. The apparatus of clause 105, wherein the cannula tip is a 33 gauge cannula tip.

Clause 108. The apparatus of any of clauses 96-106, wherein the second end comprises a one-way check valve integrated in the hub that prevent back flow.

Clause 109. The apparatus of clause 108, wherein the valve is further connected to tubing.

Clause 110. The apparatus of any of clauses 101-109, wherein the cannula tip has an outer diameter range of about 0.30 mm to about 0.18 mm.

Clause 111. The apparatus of any of clauses 101-110, wherein the cannula tip is made of a flexible polyimide material or metal.

Clause 112. The apparatus of any of clauses 101-111, wherein the cannula tip has a length of 1 mm, 2 mm, 3 mm, 4 mmm, or 5 mm.

Clause 113. The apparatus of any of clauses 101-112, wherein the cannula tip is flexible.

Clause 114. The apparatus of any of clauses 101-113, wherein the cannula tip is rigid.

Clause 115. The apparatus of any of clauses 101-114, wherein the cannula is attached to an injection device.

Clause 116. The apparatus of any of clauses 101-115, wherein the injection device is a syringe.

Clause 117. The apparatus of clause 116, wherein the syringe comprises a handle to adjust the flow path of fluid.

Clause 118. The apparatus of clause 116 or 117, wherein the syringe is a microinjection syringe.

Clause 119. The apparatus of any of clauses 116-118, wherein the syringe comprises a fluid volume of about 1 mL.

Clause 120. The apparatus of any of clauses 101-119, wherein the tubular hub comprises a fluid volume less than 200 μL

Clause 121. The apparatus of any of clauses 101-119, wherein the tubular hub comprises a fluid volume less than 100 μL

Clause 122. The apparatus of any of clauses 101-121, wherein the tubular hub comprises a fluid volume of about 50 μL.

Clause 123. The apparatus of any of clauses 101-122, wherein the cannula apparatus is further defined as a subretinal delivery apparatus.

Clause 124. The apparatus of any of clauses 101-123 for use in the delivery of cells to a tissue of a subject.

Clause 125. The apparatus of any of clauses 101-123 for use in the treatment of an ocular condition in a subject comprising administering an effective amount of cells to an eye of said subject.

Clause 126. The use of clause 125, wherein the cells are loaded into the cannula and injected to the eye of said subject with the same cannula tip.

Clause 127. The use of clause 125, wherein the cannula tip is not changed between loading into the cannula and injected to the eye of said subject.

Clause 128. The use of clause 125, wherein the cells are delivered from the injection attachment at a controlled pressure.

Clause 129. The use of any of clauses 126-128, wherein the cells are not redistributed prior to loading to the injection attachment.

Clause 130. The use of any of clauses 126-128, wherein the cells are redistributed prior to loading to the injection attachment.

Clause 131. The use of clause 130, wherein the cells are redistributed by vortexing or manual agitation.

Clause 132. The use of any of clauses 126-131, wherein the cells are injected subretinally.

Clause 133. The use of any of clauses 126-132, wherein the cells are further defined as cell aggregates.

Clause 134. The use of any of clauses 126-133, wherein the cells are further defined as single cells.

Clause 135. The use of any of clauses 126-134, wherein the cells are in a formulation buffer.

Clause 136. The use of clause 135, wherein the formulation buffer is a balanced salt solution.

Clause 137. The use of clause 136, wherein the balanced salt solution further comprises benzonase and/or human serum albumin.

Clause 138. The use of any of clauses 133-137, wherein the cell aggregates are not in suspension when injected to the tissue of said subject.

Clause 139. The use of any of clauses 133-138, wherein the cells are present in the cannula for at least 5 minutes between loading and injecting to allow aggregate settling in the cannula hub.

Clause 140. The use of any of clauses 133-138, wherein the cells are present in the cannula for at least 5 minutes between loading and injecting to allow cell settling in the cannula hub.

Clause 141. The use of any of clauses 133-140, wherein the cannula is pointed down to allow cell setting in the cannula hub.

Clause 142. The use of any of clauses 133-139, wherein the cell aggregates are photoreceptor precursor cell aggregates.

Clause 143. The use of any of clauses 126-138, wherein the cells are retinal progenitor cells and/or photoreceptor precursor cells.

Clause 144. The use of any of clauses 133-138, wherein the cell aggregates are retinal progenitor cells and/or photoreceptor precursor cells.

Clause 145. The use of any of clauses 126-142, wherein the cells are injected in a volume of less than 200 μL.

Clause 146. The use of any of clauses 126-145, wherein the cells are injected in a volume less than 100 μL.

Clause 147. The use of any of clauses 126-146, wherein the cells are injected in a volume of about 50 μL.

Clause 148. The use of any of clauses 126-147, wherein at least 1 million cell are injected.

Clause 149. The use of any of clauses 126-148, wherein at least 2 million cells are injected.

Clause 150. The use of any of clauses 126-149, wherein at least 25% of the cells that are loaded into the injection attachment are injected to the eye of said subject.

Clause 151. The use of any of clauses 126-150, wherein the ocular condition is inherited retinal disease, age-related macular degeneration (AMD), inherited macular degeneration, Stargardt's macular dystrophy, Best disease, choroideremia, diabetic retinopathy, retinal vascular disease, damage caused by retinopathy pf prematurity (ROP), or viral infection of the eye.

Clause 152. A kit comprising the apparatus of any of clauses 101-123 and cells.

Clause 153. A method for preparing a cell composition for delivery comprising:
(a) concentrating the cell composition;
(b) preparing a bulk dose of said cell composition;
(c) aliquoting a master dose from said bulk dose; and
(d) dispending said aliquoted dose.

Clause 154. The method of clause 153, wherein the cell composition is a cryopreserved cell composition.

Clause 155. The method of clause 153, further comprising thawing and resuspending the cryopreserved cell composition prior to step (a).

Clause 156. The method of any of clauses 153-155, further comprising dissociating and counting the cells in the cell composition after preparing the bulk dose.

Clause 157. The method of any of clauses 153-156, wherein step (c) comprises adding formulation buffer to reach a target concentration.

Clause 158. The method of any of clauses 153-157, wherein the bulk dose is 75 μL.

Clause 159. The method of any of clauses 153-158, wherein the aliquoted dose is 50 μL.

Clause 160. The method of any of clauses 153-159, wherein the cell composition is resuspended in a formulation buffer.

Clause 161. The method of any of clause 160, wherein the formulation buffer comprises a balanced salt solution.

Clause 162. The method of clause 160 or 161, wherein the formulation buffer further comprises albumin.

Clause 163. The method of clause 162, wherein the albumin is human serum albumin.

Clause 164. The method of any of clauses 153-163, further comprising loading the cell composition in an apparatus of any of clauses 96-123.

Clause 165. The method of any of clauses 153-163, further comprising delivering the cell composition according to the method of any of clauses 1-95.

### ****

All methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### References

The following references, to the extent that they provide exemplary procedural or other details supplementary to those set forth herein, are specifically incorporated herein by reference.
U.S. Patent Publication No. 2002/0076747
International Patent Publication No. WO 98/30679
Remington's The Science and Practice of Pharmacy, 21^{st} Edition, A R Gennaro (Lippincott, Williams & Wilkins, Baltimore, MD, 2006
Sambrook and Russel, Molecular Cloning: A Laboratory Manual, 3rd Ed. Cold Spring Harbor Lab. Press, 2001.

What is claimed is:

1. A method of cell transplantation using an injection attachment having a hub containing a plurality of cells and a cannula extending from a distal end of the hub, the method comprising:
orienting the injection attachment such that the cannula points downwardly until the plurality of cells settle together proximate the distal end;
inserting the cannula into a tissue of a subject; and
injecting the plurality of cells into the tissue of the subject using the injection attachment.

2. The method of claim 1, wherein the hub includes a funnel shaped inner surface extending between the distal end and a proximal end.

3. The method of claim 2, wherein the inner surface tapers inward from the proximal end to the distal end such that a diameter of the inner surface is smaller at the distal end than the diameter of the inner surface at the proximal end.

4. The method of claim 2, wherein the inner surface of the hub does not overlap with the cannula.

5. The method of claim 1, wherein the injection attachment is oriented such that the cannula points downwardly for at least 30 seconds.

6. The method of claim 1, wherein the injection attachment is oriented such that the cannula points downwardly for at least 2 minutes.

7. The method of claim 1, wherein the injection attachment is oriented such that the cannula points downwardly for at least 5 minutes.

8. The method of claim 1, wherein at least 99% of the plurality of cells within the hub are injected into the tissue.

9. The method of claim 1, wherein the injection attachment is held at an angle greater than 45 degrees relative to a ground surface while injecting the plurality of cells into the tissue of the subject.

10. The method of claim 1, further comprising:
providing a plurality of cells to the injection attachment such that the hub is prefilled.

11. The method of claim 10, wherein the plurality of cells are drawn in through the distal end of the cannula to the hub prior to an injection and injected through the cannula.

12. The method of claim 11, wherein the cannula is not changed between providing the plurality of cells into the hub and injecting the cells into the tissue of the subject.

13. The method of claim 1, wherein the cannula comprises a cannula shaft and a cannula tip extending from a distal end thereof.

14. The method of claim 13, wherein the cannula tip is a 33 gauge cannula tip.

15. The method of claim 1, wherein the cannula tip is blunt ended.

16. The method of claim 1, wherein the tissue is retinal tissue.

17. The method of claim 1, wherein the cells are stem cells or stem cell derived cells.

18. A method of cell transplantation in an eye using an injection attachment having a hub containing a plurality of cells and a cannula extending from a distal end of the hub, the method comprising:
orienting the injection attachment such that the cannula points downwardly until the plurality of cells settle together proximate the distal end;
inserting the cannula into retinal tissue of a subject; and
injecting the plurality of cells into the retinal tissue of the subject using the injection attachment,
wherein the injection attachment is oriented such that the cannula points downwardly for at least 2 minutes,
wherein the hub includes an engagement end and a delivery end and an inner surface extending between the engagement end and the delivery end,
wherein the inner surface tapers inward between the engagement end to the delivery end.

19. The method of claim 18, wherein the plurality of cells is provided to the hub and injected through the cannula.

20. An injection attachment for cell transplantation in an eye, comprising:
a hub having a proximal end, a distal end, and an inner surface extending therebetween defining an inner volume; and
a cannula coupled to the distal end of the hub in fluid communication with the inner volume,
wherein the inner volume contains a prefilled volume of cells,
wherein the inner volume is a generally funnel shape,
wherein the inner surface of the hub does not overlap with the cannula, and
wherein at least 90% the cells settle proximate the distal end of the hub when the distal end of the hub points downward.

21. The injection attachment of claim 20, wherein the hub comprises:
a body defining the proximal end of the hub; and
a bushing coupled to an inner surface of the body and defining the distal end of the hub.

22. The injection attachment of claim 20, wherein the inner surface tapers inward from the proximal end to the distal end such that a diameter of the inner surface is smaller at the distal end than the diameter of the inner surface at the proximal end, and
wherein the inner volume does not retain cells after an injection.
